(19) United States
(12) Patent Application Publication
(10) Pub. No.: US 2016/0161502 A1
(43) Pub. Date: Jun. 9, 2016

(54) AFFINITY CAPTURE OF CIRCULATING BIOMARKERS

(71) Applicant: Aethlon Medical, Inc., San Diego, CA (US)

(72) Inventors: R. Paul Duffin, San Diego, CA (US); James Joyce, San Diego, CA (US); Richard H. Tullis, Encinitas, CA (US)

(21) Appl. No.: 14/790,684

(22) Filed: Jul. 2, 2015

Related U.S. Application Data
(63) Continuation of application No. 14/512,129, filed on Oct. 10, 2014, now abandoned.
Application No. 14/512,129 is a continuation of application No. 13/351,166, filed on Jan. 16, 2012, now abandoned.
Application No. 13/351,166 is a continuation of application No. PCT/US2009/066626, filed on Dec. 3, 2009.
Application No. 13/351,166 is a continuation of application No. 13/131,860, now abandoned.
(60) Provisional application No. 61/119,990, filed on Dec. 4, 2008.
(60) Provisional application No. 61/119,990, filed on Dec. 4, 2008.

(51) Int. Cl. G01N 33/68

(52) U.S. Cl. CPC G01N 33/6893; G01N 33/6827; G01N 2333/4709; G01N 2800/2814

(57) ABSTRACT

Methods, devices and systems for capturing biomarkers are provided. In particular, methods, compositions, and systems that utilize affinity capture devices comprising a processing chamber, affinity capture agent and porous membrane are provided.

### CROSS REFERENCE TO RELATED APPLICATIONS

This application is a continuation of U.S. application Ser. No. 14/512,129, filed Oct. 10, 2014, which is a continuation of U.S. application Ser. No. 13/351,166, filed Jan. 16, 2012, which is a continuation of International Patent Application No. PCT/US2009/066626, filed Dec. 3, 2009, which claims priority to U.S. Provisional Application No. 61/119,990, filed Dec. 4, 2008 and U.S. application Ser. No. 13/351,166 is a continuation of U.S. application Ser. No. 13/131,860, filed May 27, 2011, which is the U.S. National Phase under 35 U.S.C. §371 of International Application No. PCT/US2009/066626, filed Dec. 3, 2009, which claims priority to U.S. Provisional Application No. 61/119,990, filed Dec. 4, 2008. The disclosures of all the foregoing are hereby expressly incorporated herein by reference in their entireties.

### FIELD OF THE INVENTION

Methods, devices and systems for capturing biomarkers are provided. In particular, methods, compositions, and systems that utilize affinity capture devices comprising a processing chamber, affinity capture agent and porous membrane are provided.

### BACKGROUND OF THE INVENTION

Despite advances in our understanding of cancer and the development of new therapeutics, cancer remains the number two killer in the US with mortality rates of many cancers remaining relatively unchanged for decades. For example, prostate cancer is the most common cancer in men, and second leading cause of death in Western countries. And while screening using markers such as prostate specific antigen (PSA) has been a valuable for early detection of prostate cancer, PSA testing currently suffers from several limitations including lack of specificity and inability to accurately predict disease progression (Stephan, C., et al., PSA and new biomarkers within multivariate models to improve early detection of prostate cancer. Cancer Lett, 2007. 249(1):18-29). In another example, ovarian cancer is the most lethal gynecological cancer in the world. Most newly diagnosed patients suffer from advanced disease and have a poor prognosis with 5-year survival rates of around 35% (Canevari, S., et al., Molecular predictors of response and outcome in ovarian cancer. Crit Rev Oncol Hematol, 2006. 60(1):19-37). Screening for ovarian cancer relies upon transvaginal ultrasonography and serum CA125 levels. Some traditional methods have low sensitivity to CA- and high false-positive rates.

In addition, cancer cells develop increasingly aggressive phenotypes that diminish the effectiveness of current treatments. The ability of cancers to evade immune detection and the development of chemotherapy resistant cells are particularly troublesome. The immunoevasive strategies used by cancer cells effectively mute the body's own defense system thereby eliminating a key element in effective cancer therapeutics. Many of these aggressive properties are manifested by the shedding of proteins, cells and membrane vesicles into the general circulation, thereby creating systemic consequences (Zhang, H. G., et al., Curcumin reverses breast tumor exosomes mediated immune suppression of NK cell tumor cytotoxicity. Biochim Biophys Acta, 2007. 1773(7):1116-23; Janowska-Wieczorek, A., et al., Microvesicles derived from activated platelets induce metastasis and angiogenesis in lung cancer. Int J Cancer, 2005. 113(5):752-60; Taylor, D. D. and C. Gercel-Taylor, Tumour-derived exosomes and their role in cancer-associated T-cell signalling defects. Br J Cancer, 2005. 92(2):305-11; Keryer-Bibens, C., et al., Exosomes released by EBV-infected nasopharyngeal carcinoma cells convey the viral latent membrane protein 1 and the immunomodulatory protein galectin 9. BMC Cancer, 2006. 6:283; Whiteside, T. L., Tumour-derived exosomes or microvesicles: another mechanism of tumour escape from the host immune system? Br J Cancer, 2005. 92(2):209-11; Yu, X., S. L. Harris, and A. J. Levine, The regulation of exosome secretion: a novel function of the p53 protein. Cancer Res, 2006. 66(9):4795-801; Keller, S., et al., Exosomes: from biogenesis and secretion to biological function. Immunol Lett, 2006. 107(2):102-8; Mears, R., et al., Proteomic analysis of melanoma-derived exosomes by two-dimensional polyacrylamide gel electrophoresis and mass spectrometry. Proteomics, 2004. 4(12):4019-31).

Some candidate biomarkers that may be useful for the diagnosis and/or prognosis of diseases and disorders may be present in biological samples, such as blood at low concentrations. Such concentrations may be too low for traditional methods of diagnosis that can include techniques such as PCR. Accordingly, there is a need to develop reliable methods that can enrich samples for particular markers for the early detection of diseases and disorders, such as cancer, e.g., prostate cancer and ovarian cancer. In addition, there is a need to identify more markers for the diagnosis and prognosis of diseases and disorders including cancer.

### SUMMARY OF THE INVENTION

Methods, devices and systems for capturing biomarkers are provided. In particular, methods, compositions, and systems that utilize affinity capture devices comprising a processing chamber, affinity capture agent and porous membrane are provided.

Some embodiments include systems for facilitating diagnostic identification of biomarkers in a biological medium. Some such systems can include an affinity capture device that includes a processing chamber configured to receive the biological medium, an affinity capture agent disposed within the processing chamber, and a porous membrane. In some embodiments, the membrane is configured such that when the biological medium is disposed in the processing chamber, biomarkers present in the medium pass through the membrane and contact the agent and are captured on the agent.

In some systems, the biological medium can include blood, urine, sputum, semen, tissue extract, and cell culture medium.

In some systems, the biomarker is a viral particle. In more such systems, the viral particle is HIV or Hepatitis C.

In some systems, the biomarker includes an antibody, antigen, protein, or aptamer. In some systems, the biomarker is a tumor biomarker that can include prostate specific antigen (PSA), prostate specific membrane antigen (PSMA), early prostate cancer antigen-1 (EPCA-1), early prostate cancer antigen-2 (EPCA-2), CA-125, B-HGG, CA-19-9, carcioembryonic antigen (CEA), EGFR, KIT, ERB2, Cathepsin D, human kallikrein 2 (hK2), alpha-methylacyl coenzyme A racemase (AMACR), galectin-3, hepsin, macrophage inhibitory cytokine (MIC-1), and insulin-like growth factor binding protein 3 (IGFBP3). In some systems, the biomarker is a brain trauma biomarker associated with Chronic Traumatic Encephalopathy (CTE).

In some systems, the biomarker is a cancer-associated exosome.

In some systems, the affinity capture agent includes a lectin, e.g., Galanthus nivalis agglutinin (GNA).

In some systems, the affinity capture agent includes an antibody or fragment thereof.

In some systems, the membrane is a hollow fiber membrane.

In addition to the foregoing systems, some embodiments of the present invention relate to methods for capturing, selectively concentrating, and harvesting exosomes and fragments thereof for use in diagnostics. Some such methods include passing a medium that includes a relatively low concentration of exosomes or fragments thereof through at least one affinity capture device. In some such methods, the affinity capture device can include a processing chamber configured to receive the medium and an affinity capture agent disposed within the processing chamber, and a porous membrane. Some methods also include selectively concentrating the exosomes and fragments thereof on the membrane by disposing the medium in the processing chamber; wherein the exosomes or fragments thereof present in the medium pass through the membrane and contact the affinity capture agent and are captured thereto. More methods also include purifying the exosomes or fragments thereof on the membrane. More methods also include harvesting the exosomes or fragments thereof from the affinity capture device.

In some methods, the exosomes or fragments thereof are cancer-specific exosomes.

In some methods, the affinity capture agent includes a lectin, e.g., GNA.

In some methods, the affinity capture agent includes an antibody.

In some methods, the exosomes or fragments thereof include a biomarker. In some such methods, the biomarker is a viral particle or fragment thereof. In more such methods, the viral particle is HIV, HCV, or CMV.

In some methods, the biomarker is a tumor biomarker that can include FasL, MMP-2, MMP-9, MHC I, or PLAP. In more methods, the biomarker includes (3-amyloid protein.

In some methods for capturing, selectively concentrating, and harvesting exosomes and fragments thereof for use in diagnostics, the exosomes are intact when harvested.

In some methods, the harvesting can also include eluting the exosomes from the affinity capture device with mannose. In more methods the harvesting can also include eluting the exosomes from the affinity capture device by lowering the pH on said membrane.

Some methods can also include identifying the harvested exosomes or fragments thereof through PCR amplification and/or through determining the identity of protein or protein fragments of said exosomes or fragments thereof.

In some methods, the medium can include blood, urine, sputum, seminal fluid, cell culture medium, or tissue extract.

In some methods, the purification step can also include reducing the complexity of the medium.

### BRIEF DESCRIPTION OF THE DRAWINGS

FIG. 1 is a schematic illustration of a longitudinal cross section of an embodiment of an affinity cartridge.

FIG. 2 is a schematic illustration of a horizontal cross section at plane 2 in FIG. 1.

FIG. 3 is an illustration of a channel from FIG. 2.

FIG. 4 is a schematic diagram of an affinity capture device.

FIG. 5A shows a graph of consensus classifiers according to Stephenson et al applied to their training set of prostate cancer cases. FIG. 5B shows a graph of consensus classifiers according to Lou et al applied to their training set of prostate cancer cases.

FIG. 6 shows a schematic diagram of a tumor secreted exosomes.

FIG. 7A shows a graph of HIV-1 envelope glycoprotein gp120 concentration in tissue culture supernatant, control PBS, and tissue culture medium circulated over a HEMOPURIFIER®. FIG. 7B shows the removal rate of gp120 by extracorporeal filtration. The system utilizes a MicroKros hollow-fiber column containing a mixture of anti-gp120 monoclonal antibodies at 50 μg/ml cross-linked to protein G agarose with DSS.

FIG. 8 shows a graph of a comparison of HIV removal rate from cell culture media, plasma and blood. Fifteen ml of the liquids were recirculated over a 1 ml affinity hemofiltration column (Q=0.9 ml/min, 37° C., pump: 1 rpm, Pharmed 6485-16 tubing). Curve is exponential best fit. Viral clearance t1/2=2.8 hours.

FIG. 9 shows a photograph of a SDS-PAGE gel that shows binding of tumor-derived exosomes by the HEMOPURIFIER® affinity capture GNA cartridges. Chromatographically isolated exosomes were applied to the cartridges in buffer (original) and the flow-through was collected. The bound exosomes were eluted from the cartridges in an equal volume of 1× Laemmli sample buffer.

FIG. 10 shows a Western blot that shows binding of tumor-derived exosomes by the HEMOPURIFIER® GNA cartridges. Chromatographically isolated exosomes from two ovarian cancer patients were applied to the cartridges in TBS (original) and the flow-through was collected. The bound exosomes were eluted from the cartridges in an equal volume of 1× Laemmli sample buffer. The expression of the tumor associated exosomal marker, EpCAM, was assayed by western immunoblotting.

FIG. 11 shows a photograph of a SDS-PAGE gel with samples from 3 ovarian cancer patients including material eluted from a HEMOPURIFIER® GNA cartridge subsequent to recirculating unfractionated ascites over the HEMOPURIFIER® (Hemopurifier), and material obtained using high exclusion limit chromatography to isolate exosome from the same ascites sample (Chrom).

FIG. 12 shows a photograph of a SDS-PAGE gel with samples including diluted plasma and material eluted from a HEMOPURIFIER® subsequent to recirculating plasma over the Hemopurifier.

FIG. 13 shows a graph for blood chemistry results of blood samples before and after recirculating the blood over a HEMOPURIFIER®.

FIG. 14 shows a graph of fluorescence over time for the elution of fluorescently-labeled mannan beads from a lectin affinity matrix.

### DETAILED DESCRIPTION

Methods, devices and systems for capturing biomarkers are provided. In particular, methods, compositions, and systems that utilize affinity capture devices are provided. Some embodiments of the present invention relate to the use of affinity capture technology as a diagnostic tool. For example, some embodiments relate to systems for facilitating diagnostic identification of biomarkers in a biological medium. Some such systems can include the use of an affinity capture device. Some affinity capture devices include a processing chamber configured to receive a biological medium, an affinity capture agent disposed within the processing chamber, and a porous membrane. In some embodiments, the porous membrane can be configured such that when the biological medium is disposed in the processing chamber, biomarkers present in the medium pass through the membrane and contact the agent and are captured on the membrane.

More embodiments of the present invention relate to methods for capturing biomarkers in a biological medium for use in diagnostic applications. For example, the biomarkers described further herein can be used in the diagnosis and/or prognosis of diseases and disorders that include examples such as cancer, such as prostate cancer, ovarian cancer, liver cancer, testicular cancer, pancreatic cancer, colon cancer, breast cancer. More examples include Alzheimer's disease, brain trauma, such as chronic traumatic encephalopathy (CTE), gastrointestinal stromal tumor, and viral infections such as HIV, HCV, and CMV.

The use of biomarkers from biological media such as biological fluids e.g., urine, blood, serum, sputum, semen, saliva, as well as biological extracts such as tissue extracts and cell culture medium, has many advantages. For example, cancers including solid tumors shed/secrete biomarkers such as macromolecules, cell vesicles, exosomes, and cells into surrounding bodily fluids. Similarly, infectious viruses shed biomarkers such as macromolecules as well as viral particles into surrounding bodily fluids. In many cases, the levels of such biomarkers can indicate the presence of a disease or disorder and/or the level of progression of the disease or disorder. In addition, the use of biological media can offer a non-invasive method to measure biomarkers, and can complement information gained from tissue biopsies. However, it will be appreciated that many useful biomarkers can be present at low concentrations in particular biological media. Moreover, useful biomarkers can be a single component within a complex mixture of materials. Accordingly, one challenge in utilizing biomarkers from biological media is the enrichment and/or isolation of low concentrations of such biomarkers from complex mixtures.

Diagnostic procedures can be limited by the sensitivity of the technique employed and the blood volume available. Some methods to detect biomarkers include the use of techniques such as the polymerase chain reaction (PCR). Such techniques may detect a few molecules per ml of sample. For example, a typical diagnostic for HIV might use a 1 ml blood sample in which the limit of detection may be 50 virus particles/ml (cpm).

However, when the virus is undetectable by this method, there may be up to 250,000 HIV particles circulating in blood (assuming a 5 liter total blood volume). Thus, even with a method as sensitive as PCR, detection is limited by sample size which is typically restricted to the destructive testing of a few ml of blood. A method to enrich/concentrate a biomarker directly from the patient or from larger volumes of donated blood would increase the sensitivity of the test significantly.

Some strategies to enrich for particular biomarkers in a complex mixture have utilized affinity chromatography to deplete abundant proteins like albumin and immunoglobulins prior to analysis. Other strategies can enrich low abundant proteins using affinity chromatography, but can require some knowledge regarding the nature of the selected biomarker (Zhang, H., et al., High throughput quantitative analysis of serum proteins using glycopeptide capture and liquid chromatography mass spectrometry. Mol Cell Proteomics, 2005. 4(2):144-55; Liu, T., et al., Human plasma N-glycoproteome analysis by immunoaffinity subtraction, hydrazide chemistry, and mass spectrometry. J Proteome Res, 2005. 4(6):2070-80; Zhou, H., J. D. Watts, and R. Aebersold, A systematic approach to the analysis of protein phosphorylation. Nat Biotechnol, 2001. 19(4):375-8, incorporated by reference in their entireties).

Many putative biomarkers have failed to be validated as useful diagnostic or prognostic indicators in clinical settings, which may be due in part to the low concentration of some such putative markers in sample biological media. For example, very few reliable serum cancer biomarkers have been shown to have clinical significance (Zhang, Z., et al., Three biomarkers identified from serum proteomic analysis for the detection of early stage ovarian cancer. Cancer Res, 2004. 64(16):5882-90). The two most widely used markers, PSA for prostate cancers, and CA-125 for ovarian cancer, have poor sensitivity and specificity. Other biomarker screens include alpha-fetoprotein for liver cancer, B-HGG for testicular cancer, CA-19-9 for pancreatic cancer, carcioembryonic antigen (CEA) and EGFR for colon cancer, KIT for gastrointestinal stromal tumor (GIST), and ERB2 for breast cancer.

Accordingly, there is a need for affinity capture technologies to selectively isolate and enrich/concentrate components of complex mixtures of biological media. Such enriched components are useful to identify new biomarkers with low abundance in particular biological media. In addition, affinity capture technologies can be useful to selectively isolate and enrich/concentrate known biomarkers for use in applications such as the diagnosis and/or prognosis of particular diseases and disorders.

In some embodiments, methods and compositions to identify low level biological markers characteristic of cancers or infectious diseases are provided.

In some embodiments, an affinity capture device and uses thereof are provided. Examples of uses for such devices include: 1) the selective concentration and detection of low level tumor specific and viral biological markers not normally detected by standard blood or biological media tests, 2) the identification of patients as candidates for blood purification therapy through diagnostic identification of deleterious immunosuppressive activity, 3) the identification and clearance of low levels of drug resistant viral strains, and, 4) the use of the blood purification therapy and related technology as a barometer of tumor progression or as a prognostic screening test for the recurrence of tumor growth.

Some embodiments of the technology described herein are designed to effectively “reset the immunological clock.” For viral infections, this action is accomplished by removing immunosuppressive viral glycoproteins and defective viral particles in conjunction with antiviral drug treatments. For cancer, immune reactivation is accomplished by removing the tumor mediated immunosuppressive activity in a biological medium in conjunction with surgical removal of a primary tumor. This can eliminate the opportunity for metastatic proliferation and growth by providing a less-permissive vascular environment. Ovarian cancer patients, for example, are known to enjoy a better prognosis with activated T cell infiltration which can only occur in the absence of exosomal immunosuppressive activity. Thus, assays to identify and determine the amount and activity of exosomes from individual cancer patients would be a great boon to cancer therapy.

Exosomes have been identified in a wide variety of tumor types. The exosomes identified in ovarian cancer patients are known to repress T cell expression of Jak3 kinase and CD3-zeta in T cells, preventing T cell anti-tumor responses. However, research also shows that other types of exosomes can activate beneficial, antigen-specific immunity. Thus, the predominant exosome activity of patients should be considered before the institution of any such therapy. Assays to identify the amount and suppressive activity of blood borne exosomes from cancer patients could identify candidates for exosome depletion therapy and provide a prognostic monitoring of the tumor load. Thus, use of the assays and therapy may play multiple therapeutic and diagnostic roles in the treatment of cancer in the future.

In the study of tumor specific antigens, the blood purification devices can concentrate larger blood volumes of suspected patients for known tumor markers and can be used to detect low level antigens. Cathepsin-D is elevated in many cases of ovarian cancer and low levels of α-methylacyl CoA racemase (AMCAR) precede PSA detection as a marker for prostate cancer. In such cases, affinity dialysis as described herein can concentrate the glycoprotein milieu of larger blood samples to increase antigen detection sensitivity. With such a tool, the development of more highly sensitive and tumor specific assays are likewise contemplated by the present invention.

### Affinity Capture Devices

Some embodiments of the present invention relate to affinity capture devices. Such devices include an affinity capture agent. As used herein the term “affinity capture agent” is a broad term and can refer to a material that can bind to a target. Examples of affinity capture agents include proteins such as lectins, antibodies, antigens, aptamers, and fragments thereof, as well as nucleic acids and oligosaccharides. Examples of lectins include Galanthus nivalis agglutinin (GNA), Narcissus pseudonarcissus agglutinin (NPA) and cyanovirin. Affinity capture agents may bind to a target present in a biological medium. Examples of targets include biomarkers. As used herein the term “biological medium” is a broad term and can refer to fluid samples comprising biological material. Examples of biological media include materials such as blood, blood derivatives e.g., serum. More examples include urine, sputum, semen, saliva, tissue fluid, ascites fluid, amniotic fluid, and the like. More examples of biological media include tissue extracts, and cell culture medium.

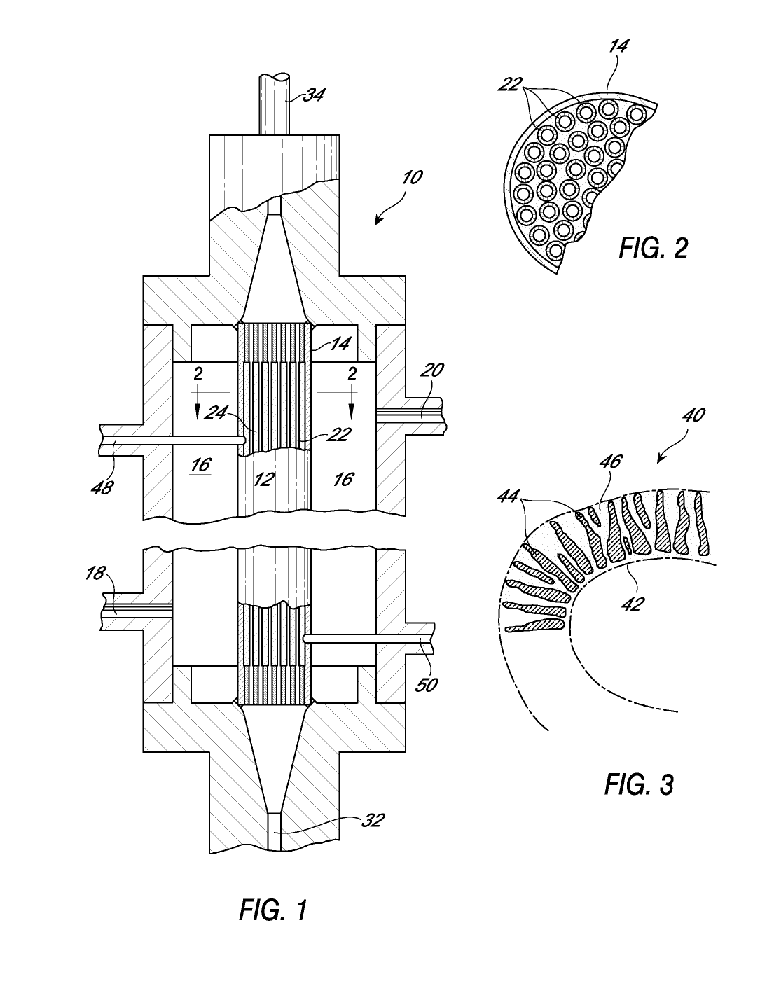

Some embodiments can utilize devices described in International Publication No. WO 2009/023332, the disclosure of which is incorporated by reference in its entirety. Some embodiments include the use of an affinity cartridge such as the device illustrated in FIG. 1 and described below in greater detail. Devices of this general type are disclosed in U.S. Pat. No. 4,714,556, U.S. Pat. No. 4,787,974 and U.S. Pat. No. 6,528,057, the disclosures of which are incorporated herein by reference in their entireties. In such devices, a biological medium can be passed through the lumen of a hollow fiber membrane, wherein an affinity capture agent is located in the extralumenal space of the cartridge, which forms a means to accept and immobilize biomarkers. Thus, the device retains biomarkers bound by the affinity capture agent while allowing other biological media components to pass through the lumen.

One embodiment of an affinity device, described in detail below with reference to FIGS. 1-3, includes multiple channels of hollow fiber membrane that forms a filtration chamber. An inlet port and an effluent port are in communication with the filtration chamber. The membrane is preferably an anisotropic membrane with the tight or retention side facing the source of a biological medium, in other words, facing the oncoming flow of a biological medium. The membrane is formed of any number of polymers known to the art, for example, polysulfone, polyethersulfone, polyamides, polyimides, and cellulose acetate. In other embodiments, the porous membrane is a sheet, rather than a channel. The sheet can be flat, or in some other configuration, such as accordion, concave, convex, conical, etc., depending on the device. In some embodiments, the membrane has pores with a mean diameter of, of about, of less than, of less than about, of more than, of more than about, 1950 nm, 1900 nm, 1850 nm, 1800 nm, 1750 nm, 1700 nm, 1650 nm, 1600 nm, 1550 nm, 1500 nm, 1450 nm, 1400 nm, 1350 nm, 1300 nm, 1250 nm, 1200 nm, 1150 nm, 1100 nm, 1050 nm, 1000 nm, 950 nm, 900 nm, 850 nm, 800 nm, 750 nm, 700 nm, 650 nm, 640 nm, 630 nm, 620 nm, 610 nm, 600 nm, 590 nm, 580 nm, 570 nm, 560 nm, 550 nm, 540 nm, 530 nm, 520 nm, 510 nm, 500 nm, 490 nm, 480 nm, 470 nm, 460 nm, 450 nm, 440 nm, 430 nm, 420 nm, 410 nm, 400 nm, 390 nm, 380 nm, 370 nm, 360 nm, 350 nm, 340 nm, 330 nm, 320 nm, 310 nm, 300 nm, 290 nm, 280 nm, 270 nm, 260 nm, 250 nm, 240 nm, 230 nm, 220 nm, 210 nm, 200 nm, 190 nm, 180 nm, 170 nm, 160 nm, 150 nm, 140 nm, 130 nm, 120 nm, 110 nm, 100 nm, 90 nm, or 85 nm, which will allow passage of macromolecules, exosomes, viral particles, and fragments thereof, but not most cellular components of a biological medium. In other embodiments, the membrane has pores in a range between any two pore diameters recited above.

In particular embodiments, the membrane can have pores 200-500 nm in diameter, more preferably, the pore size is 600 nm, which will allow passage of macromolecules, exosomes, viral particles, and fragments thereof, but not most cellular components of a biological medium, e.g., blood and blood cells (red blood cells 10,000 nm diameter, lymphocytes 7,000-12,000 nm diameter, macrophages 10,000-18,000 nm diameter, thrombocytes 1000 nm). Optionally, by selecting a pore size that is smaller than the diameter of the cellular components of a biological medium, the membrane excludes substantially all cells from passing through the pores and entering the extrachannel or extralumenal space of the device that contains the affinity capture agent. In some embodiments, a pore size is selected that is smaller than only some blood cell types.

A diagram of one embodiment of a device is shown in FIG. 1. The device comprises a cartridge 10 comprising a biological medium-processing chamber 12 formed of interior glass or plastic wall 14. Around chamber 12 is an optional exterior chamber 16. A temperature controlling fluid can be circulated into chamber 16 through port 18 and out of port 20. The device includes an inlet port 32 for the biological medium and an outlet port 34 for the effluent. The device also provides one or more ports 48 and 50, for accessing the extrachannel or extralumenal space in the cartridge. FIG. 2 is a schematic illustration of a horizontal cross section at plane 2 in FIG. 1. As shown in FIGS. 1 and 2, chamber 12 contains a plurality of membranes 22. These membranes preferably have a 0.3 mm inside diameter and 0.5 mm outside diameter. In some embodiments, the outside or inside diameter is 0.025 mm to 1 mm more preferably 0.1 to 0.5 mm more preferably 0.2 to 0.3 mm, as close to the outside diameter as allowed to minimize flow path length while still providing structural integrity to the fiber. FIG. 3 is a cross sectional representation of a channel 22 and shows the anisotropic nature of the membrane. As shown in FIG. 3, a hollow fiber membrane structure 40 is preferably composed of a single polymeric material which is formed into a tubular section comprising a relatively tight plasmapheresis membrane 42 and relatively porous exterior portion 44 in which can be immobilized affinity capture agents, e.g., lectins 46. During the operation of the device, a solution containing the affinity capture agents is loaded on to the device through port 48. The affinity capture agents are allowed to immobilize to the exterior 22 of the membrane in FIG. 2. Unbound affinity capture agents can be collected from port 50 by washing with saline or other solutions. Alternatively, the affinity capture agents can be bound to a substrate which is loaded into the extrachannel or extralumenal space, either as a dry substance (e.g. sand), or in solution or slurry.

In another embodiment, the device comprises a processing chamber having affinity capture agent disposed within the processing chamber, wherein said affinity capture agents binds biomarkers, e.g., macromolecules, viral particles, exosomes, or fragments thereof, and traps them in the processing chamber. The biological medium can directly contact the affinity capture medium. In other embodiments, the device has a porous membrane which divides the chamber into one or more portions, such that the affinity capture agent is located in only a portion of the chamber. The preferred device utilizes hollow channel fiber membranes, but one or more sheets of membranes that divide the chamber are also contemplated. Where a membrane is used, the biological medium is filtered by the membrane, such that some portion of the biological medium is excluded from the portion of the chamber containing the affinity capture agent (e.g., blood cells or other large cells which cannot pass through the pores of the membrane).

In some embodiments, the affinity capture agent can include proteins, for example, lectin, antibody, and antigen. The technology to immobilize proteins in dialysis-like cartridges has been developed (Ambrus et al., Science 201(4358): 837-839, 1978; Ambrus et al., Ann Intern Med 106(4): 531-537, 1987; Kalghatgi et al. Res Commun Chem Pathol Pharmacol 27(3): 551-561, 1980, incorporated by reference in their entireties). An illustration of preparing proteins for immobilization to the hollow fibers for the method of the present invention is presented in U.S. Pat. No. 4,714,556, U.S. Pat. No. 4,787,974, and U.S. Pat. No. 5,528,057, incorporated by reference in their entireties.

For binding of affinity capture agents, e.g., proteins, to the membrane, the polymers of the membrane are first activated, for example, made susceptible for combining chemically with proteins, by using processes known in the art. Any number of different polymers can be used. To obtain a reactive polyacrylic acid polymer, for example, carbodiimides can be used (Valuev et al., 1998, Biomaterials, 19:41-3). Once the polymer has been activated, the proteins can be attached directly or via a linker to form in either case an affinity matrix. Suitable linkers include, but are not limited to, avidin, streptavidin, biotin, protein A, and protein G. The proteins can also be directly bound to the polymer of the membrane using coupling agents such as bifunctional reagents, or can be indirectly bound. In one embodiment, the lectin, GNA, covalently coupled to agarose can be used to form an affinity matrix.

In some embodiments, a protein is attached to a substrate instead of, or in addition to, the membrane. Suitable substrates include, but are not limited to, silica (e.g. glass beads, sand, diatomaceous earth) polysaccharides (e.g. dextran, cellulose, agarose), proteins (e.g. gelatin) and plastics (e.g. polystyrenes, polysulfones, polyethersulfones, polyesters, polyurethanes, polyacrylates and their activated and native amino and carboxyl derivatives). The protein can be bound to the substrates through standard chemical means, either directly, or through linkers such as C2 to C>20 linear and branched carbon chains, as well as the plastics, other proteins and polysaccharides listed above. For most synthetic purposes, C18 is the preferred upper limit but the chains can be added together for solubility reasons. Preferred linkers include: C2 to C18 dicarboxylates, diamines, dialdehydes, dihalides, and mixtures thereof (e.g. aminocarboxylates) in both native and activated form (e.g. disuccinimidyl suberimidate (DSS)). In some embodiments, one or more substrates can be used as linkers, alone or in combination with the substances listed as linkers. For example, dextran can be attached to sand, and additional linkers can then optionally be added to the dextran.

### HEMOPURIFIER® Cartridges

Some embodiments of the devices described herein can include a HEMOPURIFIER® affinity capture cartridge (Aethlon). A HEMOPURIFIER® affinity capture cartridge can include a hollow-fiber plasmapheresis cartridge comprising affinity capture agents such as immobilized lectins, antibodies or other binding agents (e.g. peptides, oligonucleotides, oligosaccharides). Such affinity capture agents can rapidly remove molecules or particles smaller than 200 nm from a biological medium, e.g., a patient's blood. As a biological medium passes through the device, non-cellular components of the biological medium are transported through pores in the hollow fibers where they are exposed to the immobilized affinity capture agent, found outside the hollow fibers. Targets, such as proteins, exosomes or other molecules are thereby selectively removed from the biological medium. A device can include a pump. Examples of devices and systems that include pumps and may be utilized with the devices, methods, and systems described herein are described in PCT International Application No. PCT/US2009/057013, incorporated herein by reference in its entirety.

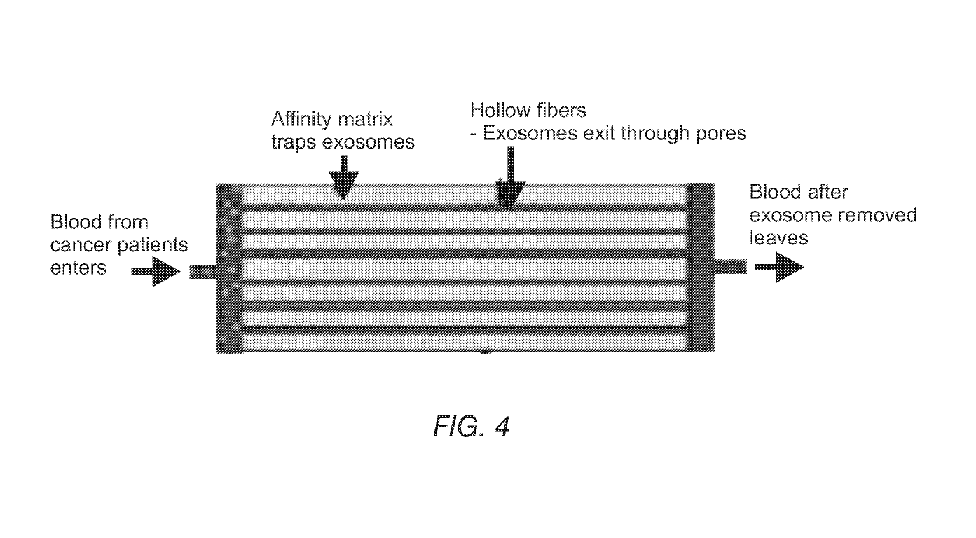

In some embodiments, a device that includes a HEMOPURIFIER® affinity capture cartridge can be a closed system (FIG. 4). In some such embodiments, affinity capture agents can be covalently immobilized preventing their release into the flow of a biological medium through the device. Such devices permit convective transport of particles below 200 nm to the outside of the hollow fiber, where an affinity bead matrix comprising affinity capture agents surround the hollow fibers, and specifically adsorbs target components from the biological medium. In embodiments that include a closed system, a pressure differential induced flux through the matrix (Starling flow) is created. This pressure differential induced flux through the matrix can prevent loss of albumin and other large molecular weight complexes in situ.

In more embodiments, devices comprising a HEMOPURIFIER® affinity capture cartridge can include one or more additional ports that allow biological medium e.g., plasma, on the outside of the hollow fibers to be removed from the cartridge, optionally with the assistance of an additional pump. In some embodiments, the affinity capture agents are located outside the cartridge in a separate affinity cartridge.

Studies have demonstrated the selective and quantitative removal of HIV derived viral proteins and particles from culture fluids, plasma, and infected blood using the HEMOPURIFIER® affinity capture cartridges in which either antibodies or lectins are utilized as an affinity capture agent (Tullis, R. H., et al., Affinity hemodialysis for antiviral therapy. I. Removal of HIV-1 from cell culture supernatants, plasma, and blood. Ther Apher, 2002. 6(3):213-20; Tullis, R. H., et al., Affinity hemodialysis for antiviral therapy. II. Removal of HIV-1 viral proteins from cell culture supernatants and whole blood. Blood Purif, 2003. 21(1):58-63; Tullis, R. H., J. A. Ambrus, Jr., and J. A. Joyce, HIV affinity hemodialysis as a treatment for AIDS. Am Clin Lab, 2001. 20(9):22-3, incorporated by reference in their entireties).

In addition, preclinical data demonstrate that lectin-based HEMOPURIFIER® affinity capture cartridges effectively bind and remove a broad spectrum of viruses including HIV, Hepatitis C virus (HCV), and Orthopox virus from human blood exploiting the polysaccharides structures common on the surface of these envelope viruses. It is also known that cancer cells are glycosylated differently compared to normal cells since cancer cells bind the high-mannose lectin Concanavalin A (ConA) while normal cells do not. Moreover, tumors routinely shed glycosylated products into circulation. Thus lectin-based HEMOPURIFIER® affinity capture cartridges are ideal for the selective removal of glycosylated tumor biomarkers from circulation. Moreover, HEMOPURIFIER® affinity capture cartridges have been proven safe in a phase I clinical trial. These studies demonstrate the capture of glycosylated hepatitis C virus (HCV) from infected end stage renal disease subjects undergoing intermittent dialysis (Table 1). Clinical chemistry and adverse event data showed that treatment with the HEMOPURIFIER® affinity capture cartridge was considered safe and well tolerated within the patient's normal hemodialysis regiment. In addition, significant amounts of HCV were captured within the HEMOPURIFIER® affinity capture cartridges.

### Systems

Some embodiments of the present invention relate to systems for facilitating diagnostic identification of biomarkers in a biological medium. Some such systems include an affinity capture device as described herein. In some embodiments, an affinity capture device can include a processing chamber configured to receive a biological medium, an affinity capture agent disposed within the processing chamber; and a porous membrane. The porous membrane can be configured such that when the biological medium is disposed in the processing chamber, biomarkers present in the medium pass through the membrane and contact the agent and are captured on the agent.

More systems include a biomarker removal system. Such systems can be utilized to remove a biomarker from an affinity capture agent for further processing. Systems for removing a biomarker can include a variety of processes. Such processes can vary with the nature of the biomarker and affinity capture agent, and the association between a biomarker and an affinity capture agent. Examples of processes that may be used with the systems described herein include changing the pH, temperature, and/or ionic concentration of the environment of the biomarker and affinity capture agent. More examples of processes include competitive elution of a biomarker from an affinity capture agent. For example, in some embodiments, sugars, e.g. mannose, can be used to elute targets bound to particular affinity capture agents such as lectins.

### Methods for Selectively Enriching Exosomes

Some embodiments of the present invention include methods for capturing, selectively concentrating, and harvesting exosomes and fragments thereof for use in diagnostics. Some such methods can include passing a biological medium comprising a relatively low concentration of exosomes or fragments thereof through at least one affinity capture device. In such embodiments, the affinity capture device can include a processing chamber configured to receive the biological medium and an affinity capture agent disposed within the processing chamber, and a porous membrane. Methods can further include selectively concentrating the exosomes and fragments thereof on the membrane by disposing the biological medium in the processing chamber, such that the exosomes or fragments thereof present in the medium pass through the membrane and contact the affinity capture agent and are captured on the affinity capture agent. More methods can also include purifying the exosomes or fragments thereof on said membrane. As used herein “purifying” is a broad term and has its ordinary meaning known in the art and can by synonymous with terms such as “enriching” and concepts such as reducing the complexity of a mixture.

The capture, isolation, harvesting and identification of exosomes from a complex biological sample, such as a biological medium that may contain components such as proteins, nucleic acids, carbohydrates and small molecules, can be a challenging task. The isolation and purification of a relatively small fraction of exosomes in relation to the vast majority of non-exosomal components in the biological samples presents a challenging task akin to the “needle-in-the-haystack” conundrum. Complexity reduction in context with exosome purification can include fractionating bona fide exosomes from a complex mixture containing non-exosomal components. The procedure of enriching/purifying exosomes from a complex biological sample allows for an increased level of sensitivity for detection purposes for diagnostic/prognostic evaluation; and also removes some or all interfering impurities such that the exosomes can be used in further applications, such as therapy.

More methods can further include harvesting the exosomes or fragments thereof from an affinity capture device for further processing in applications such as diagnosis and/or prognosis. Methods to harvest exosomes or fragments thereof from an affinity capture device are described herein and can include, for example, changing the pH, temperature, and/or ionic concentration of the environment of the exosomes or fragments thereof and affinity capture agent. More examples of methods to harvest exosomes or fragments thereof from an affinity capture device include competitive elution of exosomes or fragments thereof from an affinity capture agent. In some embodiments, intact exosomes can be harvested. In more embodiments, fragments of exosomes can be harvested.

Methods for selectively enriching exosomes can utilize the devices described herein. The term “exosome” and grammatical equivalents, is a broad term and is used herein as would be understood by a person with ordinary skill in the art. Exosomes include vesicles secreted by a wide range of eukaryotic cells, e.g., mammalian cells, such as epithelial, neural, and hematopoietic and tumor cells. The protein content of exosomes can vary with cell origin. In some methods provided herein, an affinity capture device can include an affinity capture agent such as a protein. Examples of proteins include lectins, e.g. GNA, and antibodies, e.g. antibodies specific to particular targets associated with exosomes or fragments thereof.

More methods for selectively enriching exosomes can include selectively enriching particular types of exosomes. Examples of the types of exosomes that may be selectively enriched using the methods described herein can include immunosuppressive exosomes and non-immunosuppressive exosomes. More examples include exosomes associated with a particular disease or disorder, such as Alzheimer's disease, chronic traumatic encephalopathy (CTE), an infection e.g. viral and non-viral infection, and cancer. Exosomes may be associated with a particular type of cancer, and/or particular stage of a disease or disorder, such as particular stage of a cancer.

In some methods, exosomes or fragments thereof can include a biomarker. Biomarkers that may be used with such methods are described herein. Examples include viral particles and fragments thereof, such as HIV, HCV, and CMV. More examples of viral particles and fragments thereof may be used with the methods, systems and devices described herein are described in Publication No. WO 2009/023332, incorporated herein by reference in its entirety. More examples of biomarkers include β-amyloid protein, and tumor biomarkers such as FasL, MMP-2, MMP-9, MHC I, and PLAP. Even more examples of biomarkers are described below.

### Biomarkers

Some embodiments of the present invention relate to the capture of targets using an affinity capture agent. Such targets can include biomarkers. In some embodiments, biomarkers are useful to determine a diagnosis and/or prognosis for a disease or disorder. In some embodiments, one or more markers can be used in the diagnosis and/or prognosis of a disease or disorder. Examples of diseases and disorders include cancer, such as prostate cancer, ovarian cancer, liver cancer, testicular cancer, pancreatic cancer, colon cancer, breast cancer. More examples include Alzheimer's disease, brain trauma, such as chronic traumatic encephalopathy (CTE), gastrointestinal stromal tumor, and viral and non-viral infections.

Particular glycosylated proteins can provide useful biomarkers for the devices, methods and systems described herein. For example, particular glycosylated proteins, including PSA and CA-125, are associated with cancer and are shed into a patient's serum as the cancer progresses (Przybylo, M., et al., Different glycosylation of cadherins from human bladder non-malignant and cancer cell lines. Cancer Cell Int, 2002. 2:6; Ciolczyk-Wierzbicka, D., et al., Carbohydrate moieties of N-cadherin from human melanoma cell lines. Acta Biochim Pol, 2002. 49(4):991-8; Litynska, A., et al., Comparison of the lectin-binding pattern in different human melanoma cell lines. Melanoma Res, 2001. 11(3):205-12; Drake, R. R., et al., Lectin capture strategies combined with mass spectrometry for the discovery of serum glycoprotein biomarkers. Mol Cell Proteomics, 2006. 5(10):1957-67; Gorelik, E., U. Galili, and A. Raz, On the role of cell surface carbohydrates and their binding proteins (lectins) in tumor metastasis. Cancer Metastasis Rev, 2001. 20(3-4):245-77; Chauhan, S. C., et al., Aberrant expression of MUC4 in ovarian carcinoma: diagnostic significance alone and in combination with MUC1 and MUC16 (CA125). Mod Pathol, 2006. 19(10):1386-94; De Mejia, E. G. and V. I. Prisecaru, Lectins as bioactive plant proteins: a potential in cancer treatment. Crit Rev Food Sci Nutr, 2005. 45(6):425-45, incorporated by reference in their entireties).

Some embodiments of the present invention relate to methods to identify additional biomarkers. As will be appreciated, the methods, devices and systems provided herein can be used to enrich particular types of targets in biological media. Such targets can further be identified as biomarkers that may be useful to determine the diagnosis and/or prognosis of a disease or disorder. For example, affinity capture agents with varying degrees of specificity may be utilized, e.g., affinity agents with broad specificity include lectins for N-glycoproteins. It is envisaged that a device may comprise one or more types of affinity capture agent, each with a different breadth of specificity. Such methods and devices could be used to enrich for candidate biomarkers with low abundance in a biological medium. In some embodiments, elution from these columns can be automated and combined with MALDI-TOF or SELDI TOF mass spectrometry for broad spectrum identification of biomarkers present in disease serum.

Some embodiments of the methods, devices, and systems described herein include biomarkers associated Chronic Traumatic Encephalopathy (CTE). CTE can include loss of neurons, scarring of brain tissue, collection of proteinaceous, senile plaques, hydrocephalus, attenuation of corpus callosum, diffuse axonal injury, neurofibrillary tangles and damage to the cerebellum. Microscopically, there are extensive tau-immunoreactive neurofibrillary tangles, astrocytic tangles, and spindle-shaped and threadlike neurites throughout the brain. The neurofibrillary degeneration of CTE is distinguished from other tauopathies by preferential involvement of the superficial cortical layers, irregular patchy distribution in the frontal and temporal cortices, propensity for sulcal depths, prominent perivascular, periventricular, and subpial distribution, and marked accumulation of tau-immunoreactive astrocytes (McKee et al, 2009 “Chronic traumatic encephalopathy in athletes: progressive tauopathy after repetitive head injury” J. Neuropathol Exp Neurol 68:709-35, incorporated by reference in its entirety). Deposition of β-amyloid, most commonly as diffuse plaques, occurs in fewer than half the cases. The condition may be etiologically related to Alzheimer's disease. In some embodiments, devices described herein can be used to enrich for candidate biomarkers that may be present in a sample patient with CTE. In more embodiments, biomarkers for CTE can be enriched from a biological medium for further diagnostic applications.

Glycomic profiles comparing serum from normal volunteers and prostate cancer patients has revealed several cancer-specific glycans that may be useful in the diagnosis and/or prognosis of cancers such as prostate cancer (Jankovic, M. M. and M. M. Kosanovic, Glycosylation of urinary prostate-specific antigen in benign hyperplasia and cancer: assessment by lectin-binding patterns. Clin Biochem, 2005. 38(1):58-65; Kyselova, Z., et al., Alterations in the serum glycome due to metastatic prostate cancer. J Proteome Res, 2007. 6(5):1822-32; Ohyama, C., et al., Carbohydrate structure and differential binding of prostate specific antigen to Maackia amurensis lectin between prostate cancer and benign prostate hypertrophy. Glycobiology, 2004. 14(8):671-9; Tabares, G., et al., Different glycan structures in prostate-specific antigen from prostate cancer sera in relation to seminal plasma PSA. Glycobiology, 2006. 16(2):132-45; Peracaula, R., et al., incorporated by reference in their entireties).

In another example, proteomic approaches can be used to identify candidate biomarkers. Protein signatures of cancer cells compared to non-cancer cells can be used to identify candidate biomarkers that may be used with the methods, devices, and systems described herein. For example, new markers may be discovered that identify rare variants within mixed tumor cell populations that possess enhanced tumorigenic or metastatic capabilities (Alaiya, A., M. Al-Mohanna, and S. Linder, Clinical cancer proteomics: promises and pitfalls. J Proteome Res, 2005. 4(4):1213-22; Petricoin, E. F., 3rd, et al., Serum proteomic patterns for detection of prostate cancer. J Natl Cancer Inst, 2002. 94(20):1576-8; Liotta, L. A. and E. C. Kohn, Cancer's deadly signature. Nat Genet, 2003. 33(1):10-1; Petricoin, E. F. and L. A. Liotta, Proteomic approaches in cancer risk and response assessment. Trends Mol Med, 2004. 10(2):59-64; Weigelt, B., et al., Molecular portraits and 70-gene prognosis signature are preserved throughout the metastatic process of breast cancer. Cancer Res, 2005. 65(20):9155-8; van de Vijver, M. J., et al., A gene-expression signature as a predictor of survival in breast cancer. N Engl J Med, 2002. 347(25):1999-2009; Ramaswamy, S., et al., A molecular signature of metastasis in primary solid tumors. Nat Genet, 2003. 33(1):49-54; Cho, W. C., Contribution of oncoproteomics to cancer biomarker discovery. Mol Cancer, 2007. 6:25; Rai, A. J. and D. W. Chan, Cancer proteomics: Serum diagnostics for tumor marker discovery. Ann N Y Acad Sci, 2004. 1022:286-94; Srinivas, P. R., et al., Proteomics in early detection of cancer. Clin Chem, 2001. 47(10):1901-11, incorporated by reference in their entireties).

An additional example includes identifying biomarkers for diagnosis and/or prognosis of prostate cancer. Numerous studies have been conducted in an effort to discover the “molecular signature” for prostate cancer that would enable early detection, accurate diagnosis, and monitor responsiveness to treatment (Wang, X., et al., Autoantibody signatures in prostate cancer. N Engl J Med, 2005. 353(12):1224-35; Semmes, O. J., G. Malik, and M. Ward, Application of mass spectrometry to the discovery of biomarkers for detection of prostate cancer. J Cell Biochem, 2006. 98(3):496-503; Ornstein, D. K and D. R. Tyson, Proteomics for the identification of new prostate cancer biomarkers. Urol Oncol, 2006. 24(3):231-6; Nelson, P. S., et al., Comprehensive analyses of prostate gene expression: convergence of expressed sequence tag databases, transcript profiling and proteomics. Electrophoresis, 2000. 21(9):1823-31. [pii]; Li, S., et al., Application of genomic technologies to human prostate cancer. Omics, 2006. 10(3):261-75; LaTulippe, E., et al., Comprehensive gene expression analysis of prostate cancer reveals distinct transcriptional programs associated with metastatic disease. Cancer Res, 2002. 62(15):4499-506; Bull, J. H., et al., Identification of potential diagnostic markers of prostate cancer and prostatic intraepithelial neoplasia using cDNA microarray. Br J Cancer, 2001. 84(11):1512-9; Brooks, J. D., Microarray analysis in prostate cancer research. Curr Opin Urol, 2002. 12(5):395-9; Bavik, C., et al., The gene expression program of prostate fibroblast senescence modulates neoplastic epithelial cell proliferation through paracrine mechanisms. Cancer Res, 2006. 66(2):794-802; Argani, P., et al., Discovery of new markers of cancer through serial analysis of gene expression: prostate stem cell antigen is overexpressed in pancreatic adenocarcinoma. Cancer Res, 2001. 61(11):4320-4; Ahram, M., et al., Proteomic analysis of human prostate cancer. Mol Carcinog, 2002. 33(1):9-15).

In some embodiments, the concentration of low level prostate cancer biomarkers or the identification of novel biomarkers in the HEMOPURIFIER® affinity capture device will allow for early detection, more accurate diagnosis, more accurate prediction of response to therapy and monitoring of recurrence.

### Prostate Cancer Biomarkers.

Some embodiments of the present invention relate to methods, devices and systems and biomarkers associated with prostate cancer. A number of biomarkers that are differentially regulated in prostate carcinoma have been identified. Examples include prostate-specific antigen (PSA), prostate specific membrane antigen, and human glandular kallikrein 2 (Yu, X., et al., The association between total prostate specific antigen concentration and prostate specific antigen velocity. J Urol, 2007. 177(4):1298-302; discussion 1301-2; Loeb, S., et al., Prostate specific antigen velocity threshold for predicting prostate cancer in young men. J Urol, 2007. 177(3):899-902; Gong, M. C., et al., Prostate-specific membrane antigen (PSMA)-specific monoclonal antibodies in the treatment of prostate and other cancers. Cancer Metastasis Rev, 1999. 18(4):483-90; Elgamal, A. A., et al., Prostate-specific membrane antigen (PSMA): current benefits and future value. Semin Surg Oncol, 2000. 18(1):10-6; Raaijmakers, R., et al., hK2 and Free PSA, a Prognostic Combination in Predicting Minimal Prostate Cancer in Screen-Detected Men within the PSA Range 4-10 ng/ml. Eur Urol, 2007; Diamandis, E. P. and G. M. Yousef, Human tissue kallikreins: a family of new cancer biomarkers. Clin Chem, 2002. 48(8):1198-205, incorporated by reference in their entireties).

More examples that can be used with the methods, devices, and systems described herein include circulating urokinase like plasminogen activator receptor forms that may be used alone or in combination with other prostate cancer biomarkers (hK2, PSA) to predict the presence of prostate cancer (Perambakam, S., et al., Induction of Tc2 cells with specificity for prostate-specific antigen from patients with hormone-refractory prostate cancer. Cancer Immunol Immunother, 2002. 51(5):263-70; McDevitt, M. R., et al., An alpha-particle emitting antibody ([213Bi]J591) for radioimmunotherapy of prostate cancer. Cancer Res, 2000. 60(21):6095-100; Steuber, T., et al., Free PSA isoforms and intact and cleaved forms of urokinase plasminogen activator receptor in serum improve selection of patients for prostate cancer biopsy. Int J Cancer, 2007. 120(7):1499-504, incorporated by reference in their entireties).

More biomarkers include early prostate cancer antigen-1 (EPCA-1), early prostate cancer antigen-2 (EPCA-2), AMACR, human kallikrein, macrophage inhibitory cytokine 1 (MIC-1) and prostate cancer specific autoantibodies (Stephan, C., et al., Three new serum markers for prostate cancer detection within a percent free PSA-based artificial neural network. Prostate, 2006. 66(6):651-9; Miyake, H., I. Hara, and H. Eto, Prediction of the extent of prostate cancer by the combined use of systematic biopsy and serum level of cathepsin D. Int J Urol, 2003. 10(4):196-200; Leman, E. S., et al., EPCA-2: a highly specific serum marker for prostate cancer. Urology, 2007. 69(4):714-20; Jiang, Z., et al., Discovery and clinical application of a novel prostate cancer marker: alpha-methylacyl CoA racemase (P504S). Am J Clin Pathol, 2004. 122(2):275-89; Hara, I., et al., Serum cathepsin D and its density in men with prostate cancer as new predictors of disease progression. Oncol Rep, 2002. 9(6):1379-83; Bradford, T. J., X. Wang, and A. M. Chinnaiyan, Cancer immunomics: using autoantibody signatures in the early detection of prostate cancer. Urol Oncol, 2006. 24(3):237-42, incorporated by reference in their entireties).

Additional biomarkers include biomarkers identified comparing gene expression from normal prostate tissue, BPH tissue, and PCa tissue has identified many potential genes upregulated in prostate cancer. These biomarkers include hepsin, a serine protease, alpha-methylacyl-CoA racemase (AMACR), macrophage inhibitory cytokine (MIC-1), and insulin-like growth factor binding protein 3 (IGFBP3).

### Prostate-Specific Antigen (PSA)

Prostate-specific antigen (PSA) is an ideal candidate for capture, isolation and concentration using the methods, devices and systems described herein. PSA is a glycosylated serine protease upregulated in prostate cancer. PSA is shed into the general circulation and serum PSA screening was approved as a screen for early detection of prostate cancer by the FDA in 1994. Several variations of PSA testing have been tested for their ability to improve the accuracy of PSA testing. Several studies suggest that the percentage of PSA bound to other molecules such as α1-antichymotrypsin correlates with disease progression. PSA velocity, the annual rate of PSA increase, has been used to predict patient survival following treatment (radical prostatectomy or external beam radiation) (Loeb, S., et al., Does body mass index affect preoperative prostate specific antigen velocity or pathological outcomes after radical prostatectomy? J Urol, 2007. 177(1):102-6; discussion 106; Vaisanen, V., et al., Characterization and processing of prostate specific antigen (hK3) and human glandular kallikrein (hK2) secreted by LNCaP cells. Prostate Cancer Prostatic Dis, 1999. 2(2):91-97, incorporated by reference in their entireties).

Another approach has been to examine how different isoforms of PSA can correlate to malignancy. Studies suggest that the precursor form of PSA, pro-PSA, is elevated in prostate cancer compared to BPH (Catalona, W. J., et al., Serum pro-prostate specific antigen preferentially detects aggressive prostate cancers in men with 2 to 4 ng/ml prostate specific antigen. J Urol, 2004. 171(6 Pt 1):2239-44; Catalona, W. J., et al., Serum pro prostate specific antigen improves cancer detection compared to free and complexed prostate specific antigen in men with prostate specific antigen 2 to 4 ng/ml. J Urol, 2003. 170(6 Pt 1):2181-5). Recent studies also suggest that differential glycosylation patterns of PSA may be used to distinguish prostate cancer from BPH.

Some of the methods, devices and systems described herein can be applied to PSA. PSA can bind to antibodies and lectins including Lens culinaris, Aleuria aurantia, Sambucus nigra, Mackia amurensis (MAA) and Concanavalin A (ConA).

Human Glandular Kallikrein 2 (hK2)

Human glandular kallikrein 2 (hK2) is a serine protease with 80% homology to PSA. hK2 is expressed at higher levels in prostate cancer than normal epithelium and its use as a potential prostate cancer biomarker is currently being investigated (Cloutier, S. M., et al., Substrate specificity of human kallikrein 2 (hK2) as determined by phage display technology. Eur J Biochem, 2002. 269(11):2747-54). The combination of free PSA and hK2 serum levels has prognostic significance in discriminating between mild and advanced prostate cancer in men with PSA levels>4 ng/ml<10 ng/ml. This is significant since most men in this PSA range are routinely subjected to radical prostatectomy. hK2 screening in conjunction with other parameters such as PSA and Gleason score may therefore more accurately identify patients who would benefit from watchful waiting from those requiring more radical treatment.

Like PSA, hK2 is a glycosylated protein with many different isoforms the relationship between the different isoforms of hK2 and prostate cancer progression has not yet been established. hK2 is present in the bloodstream at concentration between 1-2% that of PSA levels. The concentration of individual isoforms may be below the level of detection of current assays. Isolation of HK2 using the Aethlon HEMOPURIFIER® affinity capture system is ideal for the capture, isolation and concentration of these rare isoforms.

### Cathepsin D

Cathepsin D is an aspartyl protease involved in protein degradation and tissue remodeling. Upregulation and release of cathepsin D is implicated in promotion of tumor cell growth, angiogenesis, local release of cytokines from stromal cells, and increased degradation of extracellular matrix thereby promoting tumor cell invasion and metastasis (Laurent-Matha, V., et al., Catalytically inactive human cathepsin D triggers fibroblast invasive growth. J Cell Biol, 2005. 168(3):489-99; Mohamed, M. M. and B. F. Sloane, Cysteine cathepsins: multifunctional enzymes in cancer. Nat Rev Cancer, 2006. 6(10):764-75; Berchem, G., et al., Cathepsin-D affects multiple tumor progression steps in vivo: proliferation, angiogenesis and apoptosis. Oncogene, 2002. 21(38):5951-5). Cathepsin D-mediated proteolysis can be direct or can be indirect through the activation of a cascade of other proteases including metalloproteases and elastase. In addition, cathepsin D can contribute to the development of chemoresistant cancer cell subpopulations (Bazzett, L. B., et al., Modulation of proliferation and chemosensitivity by procathepsin D and its peptides in ovarian cancer. Gynecol Oncol, 1999. 74(2):181-7, incorporated by reference in their entireties).

Elevated expression and secretion of cathepsin D has been observed for numerous cancer types including ovarian cancer, gliomas, lung cancer, prostate cancer, colorectal cancer, breast cancer and pancreatic cancer (Skrzydlewska, E., et al., Evaluation of serum cathepsin B and D in relation to clinicopathological staging of colorectal cancer. World J Gastroenterol, 2005. 11(27):4225-9; Zhou, H., et al., Collection, storage, preservation, and normalization of human urinary exosomes for biomarker discovery. Kidney Int, 2006. 69(8):1471-6; Hegmans, J. P., et al., Proteomic analysis of exosomes secreted by human mesothelioma cells. Am J Pathol, 2004. 164(5):1807-15; Pisitkun, T., R. F. Shen, and M. A. Knepper, Identification and proteomic profiling of exosomes in human urine. Proc Natl Acad Sci USA, 2004. 101(36):13368-73; Fukuda, M. E., et al., Cathepsin D is a potential serum marker for poor prognosis in glioma patients. Cancer Res, 2005. 65(12):5190-4; Losch, A., et al., Cathepsin D in ovarian cancer: prognostic value and correlation with p53 expression and microvessel density. Gynecol Oncol, 2004. 92(2):545-52; Baekelandt, M., et al., The significance of metastasis-related factors cathepsin-D and nm23 in advanced ovarian cancer. Ann Oncol, 1999. 10(11):1335-41; Brouillet, J. P., et al., Increased cathepsin D level in the serum of patients with metastatic breast carcinoma detected with a specific pro-cathepsin D immunoassay. Cancer, 1997. 79(11):2132-6; Kristensen, G. B., et al., Evaluation of the prognostic significance of cathepsin D, epidermal growth factor receptor, and c-erbB-2 in early cervical squamous cell carcinoma. An immunohistochemical study. Cancer, 1996. 78(3):433-40, incorporated by reference in their entireties).

In many cases, expression of cathepsin D correlates with development of metastatic disease and may therefore serve as a prognostic marker of cancer progression. Higher cathepsin-D serum levels are associated with poor prognosis for numerous cancer types, including ovarian cancer, suggesting its role as a possible serum biomarker (Lou, X., et al., Cathepsin D is secreted from M-BE cells: its potential role as a biomarker of lung cancer. J Proteome Res, 2007. 6(3):1083-92; Hornung, R., et al., Analysis of potential prognostic factors in 111 patients with ovarian cancer. Cancer Lett, 2004. 206(1):97-106, incorporated by reference in their entireties).

Serum cathepsin D levels can be positively correlated with more aggressive histological grades of glioma. Cathepsin D has been implicated in prostate cancer tumor growth and elevated levels of circulating cathepsin D is elevated in men with advanced prostate cancer (Nomura, T. and N. Katunuma, Involvement of cathepsins in the invasion, metastasis and proliferation of cancer cells. J Med Invest, 2005. 52(1-2):1-9; Vetvicka, V., J. Vetvickova, and M. Fusek, Role of procathepsin D activation peptide in prostate cancer growth. Prostate, 2000. 44(1):1-7, incorporated by reference in their entireties).

Combined use of serum assays for cathepsin D and PSA or prostate tumor volume can be a useful predictor of prostate cancer progression. Lectin capture chromatography can be applied to the isolation of cathepsin D since it is a glycosylated protein capable of binding the lectins Galanthus nivalis agglutinin (GNA) and concanavalin A (ConA) (Wright, L. M., et al., Purification and characterization of cathepsin D from normal human breast tissue. J Protein Chem, 1997. 16(3):171-81). Cathepsin-D is an ideal biomarker for capture, isolation and concentration using the methods, devices, and systems described herein, including for example, using the Aethlon HEMOPURIFIER® affinity capture system, because it can be isolated by antibodies or lectins, it is present in elevated levels of prostate cancer patient sera, and has significant clinical relevance.

α-methylacyl CoA Racemase (AMACR)

Another example marker includes α-methylacyl CoA racemase (AMACR). AMACR is highly upregulated in prostate cancer tissue and is not expressed in benign tissue (Zehentner, B. K., et al., Detection of alpha-methylacyl-coenzyme-A racemase transcripts in blood and urine samples of prostate cancer patients. Mol Diagn Ther, 2006. 10(6):397-403; Sreekumar, A., et al., Humoral immune response to alpha-methylacyl-CoA racemase and prostate cancer. J Natl Cancer Inst, 2004. 96(11):834-43; Maria McCrohan, A., et al., Effects of the dual 5 alpha-reductase inhibitor dutasteride on apoptosis in primary cultures of prostate cancer epithelial cells and cell lines. Cancer, 2006. 106(12):2743-52, incorporated by reference in their entireties).

Protein expression has been validated by RT-PCR and immunohistochemistry. Elevated AMACR expression levels can be detected prior to an increase in PSA and it expression is negligible in normal prostate tissue. AMACR is therefore a very attractive candidate for the specific and early diagnosis of prostate cancer. Recent studies have established that AMACR can be detected in the serum and urine of prostate cancer patients and could be used to identify patients with metastatic disease. AMACR is among the biomarkers recently identified by the cell-specific profiling expression analysis approach of Wang et al. (Wang, Y., et al., The challenge of developing predictive signatures for the outcome of newly diagnosed prostate cancer based on expression analysis and genetic changes of tumor and non-tumor cells, in 2007 American Association for Cancer Research Annual Meeting. 2007: Los Angeles, Calif., incorporated by reference in their entireties). This biomarker together with other novel and known biomarkers are being used to assemble a predictive multigene panel.

In some embodiments, antibodies to AMACR can be provided for use as affinity capture agents in the methods, devices and systems described herein.

### Expression Analysis Reveals Genomic Signature for Relapse-Free Survival of Prostate Cancer

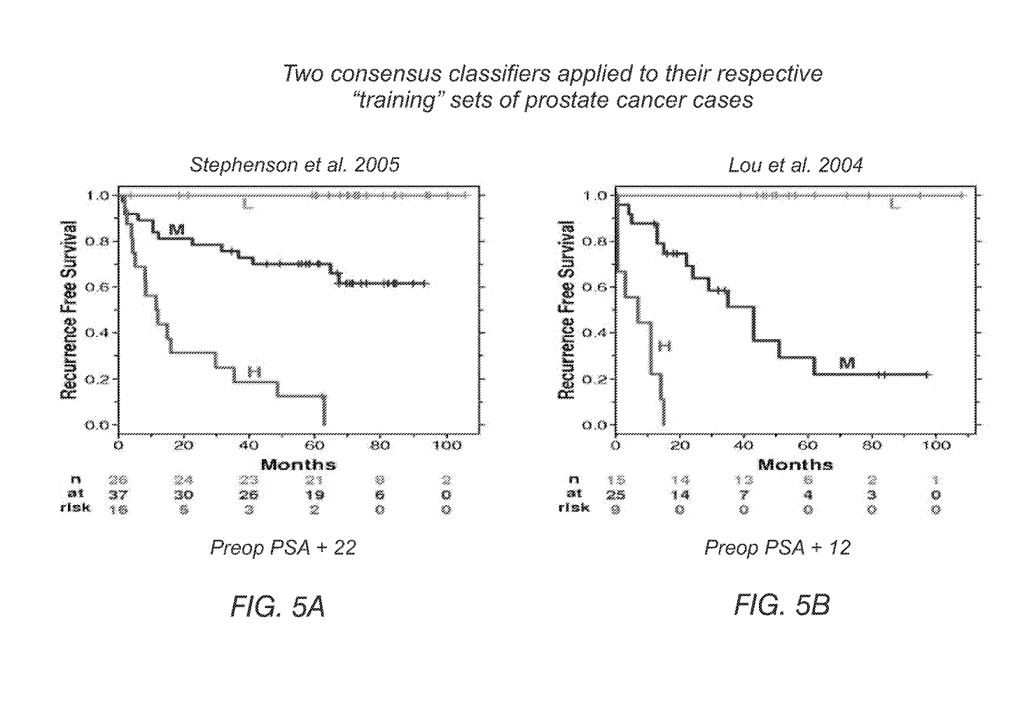

FIG. 5 shows the application of consensus classifiers trained on the expression data of a set of 79 published prostate cancer cases (left) and on a set of 49 published prostate cancer cases as Kaplan-Meier curves. (L), cases classified as low risk of relapse; (H), cases classified has high risk of relapse based on preoperative PSA; (I), cases classified as intermediate risk of relapse.

All classifications are based on analysis of gene expression data obtained from prostatectomy samples, for example, data at about the time of diagnosis. The classifiers also utilize preoperative PSA as one node of the decision tress derived by recursive partitioning, for example equivalent to the use of one gene. Thus the classifiers contain preoperative PSA plus 22 gene for Stephenson et al. and preoperative PSA plus 12 genes for Lou et al. (Stephenson, A. J., et al., Integration of gene expression profiling and clinical variables to predict prostate carcinoma recurrence after radical prostatectomy. Cancer, 2005. 104(2):290-8). No genes are shared by the two classifiers.

### Ovarian Cancer Biomarkers

Some embodiments of the present invention relate to methods, devices and systems and biomarkers associated with ovarian cancer. Ovarian cancer is the most lethal gynecological cancer in the world. Most newly diagnosed patients suffer from advanced disease and have a poor prognosis with 5-year survival rates of around 35% (Canevari, S., et al., Molecular predictors of response and outcome in ovarian cancer. Crit Rev Oncol Hematol, 2006. 60(1):19-37). Screening for ovarian cancer relies upon transvaginal ultrasonography and serum CA125 levels. Some traditional methods have low sensitivity to CA- and high false-positive rates.

### Cathepsin D

An example biomarker that can be used with the methods, devices, and systems described herein includes cathepsin D. Serum levels of Cathepsin D are elevated in ovarian cancer patients and significantly higher in patients with metastatic disease indicating that cathepsin D may be an important independent prognostic factor for patient survival. Further studies are needed to validate cathepsin D as an ovarian cancer serum biomarker. Lectin capture chromatography can be applied to the isolation of cathepsin D since it is a glycosylated protein capable of binding the lectins Galanthus nivalis agglutinin (GNA) and concanavalin A (ConA). Cathepsin-D is an ideal candidate for capture, isolation and concentration by the affinity Aethlon HEMOPURIFIER® affinity capture system because it can be isolated by antibodies or lectins, it is present in elevated levels of ovarian cancer patient sera, and has significant clinical relevance.

### Galectins

More examples of biomarker that may be used with the methods, devices, and systems described herein includes galectins. Galectins are a family of animal lectins with high binding to β-galactose oligosaccharides. Galectins are capable of binding a variety of glycoproteins and glycolipids found in the extracellular matrix and cell surface and therefore capable of modulating cell-cell and cell-matrix interactions critical in cancer progression. Galectin expression is upregulated in numerous cancers and altered galectin expression has been correlated with aggressive phenotype and acquisition of the metastatic phenotype. Although galectin-3 expression has been strongly correlated with cancer progression, serum levels of many galectins are very low and difficult to detect using current methods. Elevated galectin-3 serum levels have been reported in sera of patients with breast, gastrointestinal, lung, HNSCC, melanoma, and ovarian cancer suggesting that circulating galectin levels may serve as diagnostic and/or prognostic markers to monitor disease progression. High levels of galectin-3 are seen in patients with advanced metastatic disease.

Removal or inhibition of circulating galectin-3 can also have therapeutic effects. Circulating galectin-3 increased metastasis and cell adhesion through interaction with the Thomsen-Friedenreich disaccharide of MUC-1 and other cell-surface glycoproteins on disseminated cancer cells. Peptides and small molecules to block these interactions are being sought as therapeutic solutions. Reduction of Galectin-3 from a patient's blood might therefore provide therapeutic benefit in addition to diagnostic/prognostic importance. Aethlon HEMOPURIFIER® affinity capture cartridges can be adapted to use lectins, antibodies, or Thomsen-Friedenreich disaccharide conjugated affinity resin to remove circulating galectin-3 and improve prognosis.

### Exosomes

Some embodiments of the present invention relate to the use of exosomes and fragments thereof. Some embodiments include the use of cancer-derived exosomes. In such embodiments, cancer-derived exosomes can be a rich source of biomarkers.

Exosomes are extracellular membrane-bound vesicles produced by many cell types including epithelial, neural, and hematopoietic and tumor cells (Valenti, R., et al., Tumor-released microvesicles as vehicles of immunosuppression. Cancer Res, 2007. 67(7): 2912-5; Liu, C., et al., Murine mammary carcinoma exosomes promote tumor growth by suppression of NK cell function. J Immunol, 2006. 176(3): p. 1375-85; Zhang, H. G., et al., Curcumin reverses breast tumor exosomes mediated immune suppression of NK cell tumor cytotoxicity. Biochim Biophys Acta, 2007. 1773(7): 1116-23; Janowska-Wieczorek, A., et al., Microvesicles derived from activated platelets induce metastasis and angiogenesis in lung cancer. Int J Cancer, 2005. 113(5): 752-60; Taylor, D. D. and C. Gercel-Taylor, Tumour-derived exosomes and their role in cancer-associated T-cell signalling defects. Br J Cancer, 2005. 92(2): 305-11; Keryer-Bibens, C., et al., Exosomes released by EBV-infected nasopharyngeal carcinoma cells convey the viral latent membrane protein 1 and the immunomodulatory protein galectin 9. BMC Cancer, 2006. 6: 283; Whiteside, T. L., Tumour-derived exosomes or microvesicles: another mechanism of tumour escape from the host immune system? Br J Cancer, 2005. 92(2): 209-11; Yu, X., S. L. Harris, and A. J. Levine, The regulation of exosome secretion: a novel function of the p53 protein. Cancer Res, 2006. 66(9): 4795-801; Keller, S., et al., Exosomes: from biogenesis and secretion to biological function. Immunol Lett, 2006. 107(2): 102-8; Mears, R., et al., Proteomic analysis of melanoma-derived exosomes by two-dimensional polyacrylamide gel electrophoresis and mass spectrometry. Proteomics, 2004. 4(12): 4019-31; Andre, F., et al., Malignant effusions and immunogenic tumour-derived exosomes. Lancet, 2002. 360(9329): 295-305; Zhou, H., et al., Collection, storage, preservation, and normalization of human urinary exosomes for biomarker discovery. Kidney Int, 2006. 69(8): 1471-6; Hegmans, J. P., et al., Proteomic analysis of exosomes secreted by human mesothelioma cells. Am J Pathol, 2004. 164(5): p. 1807-15; Pisitkun, T., R. F. Shen, and M. A. Knepper, Identification and proteomic profiling of exosomes in human urine. Proc Natl Acad Sci USA, 2004. 101(36): p. 13368-73; Rajendran, L., et al., Alzheimer's disease beta-amyloid peptides are released in association with exosomes. Proc Natl Acad Sci USA, 2006. 103(30): 11172-7; Masciopinto, F., et al., Association of hepatitis C virus envelope proteins with exosomes. Eur J Immunol, 2004. 34(10): 2834-42; Taylor, D. D., S. Akyol, and C. Gercel-Taylor, Pregnancy-associated exosomes and their modulation of T cell signaling. J Immunol, 2006. 176(3): 1534-42; Fang, Y., et al., Higher-Order Oligomerization Targets Plasma Membrane Proteins and HIV Gag to Exosomes. PLoS Biol, 2007. 5(6): e158; Wieckowski, E. and T. L. Whiteside, Human tumor-derived vs dendritic cell-derived exosomes have distinct biologic roles and molecular profiles. Immunol Res, 2006. 36(1-3): 247-54; Johnstone, R. M., Exosomes biological significance: A concise review. Blood Cells Mol Dis, 2006. 36(2): 315-21, incorporated by reference in their entireties).

Exosomes are produced by the inward budding of the membrane into the lumen of endosomes creating multivesicular vesicles that are released upon membrane fusion. These exosomes contain membrane and cytosolic proteins reflective of their cell of origin. Exosomes are thought to mediate intracellular communication and may play important roles in normal and pathological processes. For example, exosomes secreted by B lymphocytes and dendritic cells serve as effective antigen presenting cells (APC) to T cells. Aberrant exosome expression has been linked to numerous pathologies (Favre, D. and B. Muellhaupt, Potential cellular receptors involved in hepatitis C virus entry into cells. Lipids Health Dis, 2005. 4(1):9). Several viruses (HCV, HIV, CMV) are thought to use exosomes to exit cells, avoid immune detection, and potentially infect other cells via cell fusion (Vingtdeux, V., et al., Alkalizing drugs induce accumulation of amyloid precursor protein by-products in luminal vesicles of multivesicular bodies. J Biol Chem, 2007. 282(25):18197-205, incorporated by reference in its entirety). Exosomes may be involved in Alzheimer's disease pathogenesis as a vehicle for export of β-amyloid proteins.

Cancer exosomes are relatively small (30-100 nm) tumor-derived membrane fragments shed by tumor cells. Exosome release by tumor cells is accelerated during cancer progression and increasing levels of tumor exosomes have been found in the blood, urine, and malignant effusions of numerous cancers. Exosome accumulation in these fluids correlates with tumor progression and has been linked to tumor aggression by promoting tumor growth, angiogenesis, metastasis and immunoevasion.

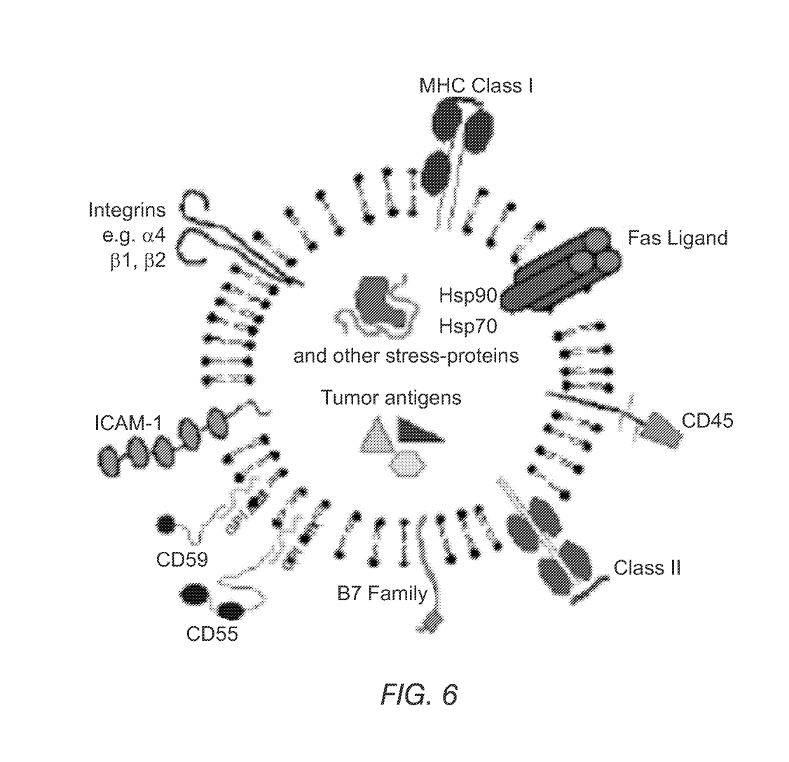

In addition, cancer-derived exosomes are enriched with both membrane and cytoplasmic proteins that mirror the specific cancer type and stage of progression (FIG. 6). For example, higher levels of circulating tumor-derived exosomes were found in patients with ovarian and endometrial cancers compared to control sera or in sera from women with benign disease (Taylor, D. D., K. S. Lyons, and C. Gercel-Taylor, Shed membrane fragment-associated markers for endometrial and ovarian cancers. Gynecol Oncol, 2002. 84(3):443-8, incorporated by reference in its entirety).

Tumor-derived membrane fragments were partially characterized and found to express FasL and the metalloproteinases, MMP-2 and MMP-9. Importantly, these markers were shown to be significantly elevated on exosomes derived from late stage cancers (Kim, J. W., et al., Fas ligand-positive membranous vesicles isolated from sera of patients with oral cancer induce apoptosis of activated T lymphocytes. Clin Cancer Res, 2005. 11(3):1010-20 incorporated by reference in its entirety).

### Tumor-Derived Exosomes and Immune Suppression

Some embodiments of the present invention relate to quantitative removal of ovarian exosomes from patients using affinity capture devices such as a HEMOPURIFIER® can also have therapeutic applications. Tumor-derived exosomes can be directly involved in tumor progression by immunosuppressive mechanisms. Tumor-derived exosomes have been shown to induce T cell apoptosis and block various aspects of T cell signaling and proliferation, cytokine production, cytotoxicity, and impair antigen presenting cell function (Taylor, D. D., et al., T-cell apoptosis and suppression of T-cell receptor/CD3-zeta by Fas ligand-containing membrane vesicles shed from ovarian tumors. Clin Cancer Res, 2003. 9(14):5113-9; Eblen, A. C., et al., Modulation of T-cell CD3-zeta chain expression in early pregnancy. Am J Reprod Immunol, 2002. 47(3):167-73). These effects are mediated, in part, by the presence of the T cell apoptosis-inducing molecule, Fas ligand, on the exosomes. In addition, tumor exosomes were shown to mediate zeta chain cleavage of the TCR-zeta chain thereby inhibited T-cell secretion of interferon gamma (Taylor, D. D., et al., Modulation of CD3-zeta as a marker of clinical response to IL-2 therapy in ovarian cancer patients. Gynecol Oncol, 2004. 94(1):54-60). Cleavage and downregulation of the TCR-zeta chain is predictive of a lack in anti-tumor responses and decreased survival in patients with a variety of cancers including ovarian, melanoma, oral carcinoma, and head and neck cancers (Kuss, I., et al., Expression of zeta in T cells prior to interleukin-2 therapy as a predictor of response and survival in patients with ovarian carcinoma. Cancer Biother Radiopharm, 2002. 17(6):631-40; Reichert, T. E., et al., The number of intratumoral dendritic cells and zeta-chain expression in T cells as prognostic and survival biomarkers in patients with oral carcinoma. Cancer, 2001. 91(11):2136-47; Zea, A. H., et al., Alterations in T cell receptor and signal transduction molecules in melanoma patients. Clin Cancer Res, 1995. 1(11):1327-35). T cell apoptosis may be mediated by direct interaction between the tumor exosome and the T cell, or indirectly by tumor exosomes binding to dendritic cells. When T cells bind dendritic cells coated with exosomes, the exosome uses dendritic cell adhesion/costimulatory molecules to form a stable interaction with the T cell and thus induces apoptosis. Indeed, it has been demonstrated that exosomes selectively bind antigen-presenting cells after in vivo injection.

Tumor-derived exosomes have been shown to suppress NK cell function in vitro and in vivo. Pre-treatment of Balb/c mice with tumor exosomes derived from different mouse tumor cell lines resulted in increased tumor growth of tumor xenografts compared to controls. Analysis revealed that the increased growth was mediated, in part, by an impaired immune response characterized by suppression of NK cell proliferation and cytotoxic activities. Further analysis demonstrated exosome-mediated inhibition of NK proliferation by decreasing cyclin D3 expression and impairment of NK cytotoxicity by interfering with Jak3-mediated release of perforin. Tumor-derived exosomes from other human cancer cell lines were shown to inhibit NK cell proliferation and cytotoxicity in a similar manner, while exosomes derived from normal cells had no effect.

In advanced cancer patients, exosomes reach higher concentrations systemically, and induction of T cell apoptosis occurs in an antigen-nonspecific, but Fos ligand, MHC I-dependent manner. The removal of tumor derived exosomes can help diminish or eliminate T cell apoptosis leading to “re-activation” of native T cell tumor immunity. Thus, the ability to isolate, concentrate, quantify, characterize, and remove cancer exosomes can identify many novel biomarkers with diagnostic and prognostic capabilities, and effectively circumvent the immunoevasiveness imposed by these particles.

### Prescreening for Immunosuppressive Exosomes

Pre-screening patients to determine that immunosuppressive exosomes are present in the blood of a tumor patient before commencing exosome depletion therapy by the methods disclosed herein will improve patient outcome. In particular, it is preferable to perform depletion therapy only in subjects who have demonstrable immunosuppressive cancer-derived exosomes. Failure to do so would presumably remove the normal exosomes (antigen presenting and immune activating exosomes) and their beneficial and normal capacity to stimulate the immune responses specific to their disease or tumor.

In some embodiments, an affinity capture device is used to collect exosomes for pre-screening patients to determine the potential therapeutic effectiveness of exosome capture and depletion. For example, because FasL induces apoptosis, a screen for exosome associated FasL could identify the concentration of exosomes in a biological medium, e.g., blood. In some embodiments, exosomes are isolated from a biological medium by density centrifugation or by affinity capture, for example using a HEMOPURIFIER® device, then an assay for FasL is performed, as described herein to identify patients most likely to benefit from removal of immunosuppressive exosomes. In another method, the fluid from the patient being evaluated is incubated in a T cell activation assay, to determine their direct suppression or killing of T-cells.

In another embodiment, a Vacutainer retrieval tube or similar device is used to draw blood for pre-screening. The blood is drawn into a plasma tube containing a matrix-lectin compound, wherein the matrix is lighter in density than the plasma separator gel. Following centrifugation, the tube would contain layers of blood cells, plasma separator gel, exosome bound, lectin matrix (e.g., GNA) and plasma. The clear plasma layer is discarded, and the plasma layer with the lectin matrix is removed and centrifuged. The lectin-exosome pellet allows resuspension of the lectin matrix in a separation buffer (e.g., Laemmli buffer or saline) for marker analysis. For example, SDS-PAGE separation of Laemmli buffer samples and Western blotting can determine the presence of transpannins, FasL or other markers of immunosuppressive exosomes. Similarly, activated T cell lines may be examined for the presence or absence of activation markers after incubation with buffered saline suspensions of suspected exosome containing samples.

### Ovarian Cancer Exosomes

Some embodiments of the present invention relate to enrichment and/or purification of ovarian cancer exosomes. Ovarian cancer exosomes are highly glycosylated and may be enriched from a biological medium using the methods, devices, and systems described herein. The concentration of exosomes in plasma of healthy volunteers is approximately between 0.5 μg/ml and <250 μg/ml. In contrast, advanced stage III ovarian cancer patients have on average 2,000 μg/ml of exosomal plasma protein with high FasL concentration therefore providing a rich exosome source. Large volumes of ascites fluids (100-200 ml) from ovarian cancer patients can be used in studies instead of peripheral blood samples. Ascites fluid is drawn as a routine procedure before surgical removal of ovarian tumors and has an exosomal content of up to 4,000-5,000 μg/ml.

Some embodiments include the isolation of subcellular particles, such as particles corresponding to exosome dimensions. Some embodiments include enriching for exosomes or particles thereof using affinity capture agents that bind targets and/or biomarkers such as MHC I, PLAP, and FasL. In some such embodiments, the affinity capture agent can comprise an antibody to the biomarker.

### Quantitative Removal of Ovarian Exosomes

Overall, exosome concentrations are much higher in cancer patients than healthy volunteers suggesting that even non-selective exosome removal may be clinically advantageous. The systemic removal of exosomes is not expected to have deleterious effects on immune responses, since naturally occurring exosomes such as T cell or dendritic cell-derived exosomes are known to act in the local lymphatic milieu and exosome concentrations in late stage ovarian cancer patients are approximately 10-fold higher than in healthy volunteers. Therefore, in addition to the diagnostic and prognostic benefits, affinity cartridges have the added benefit of selectively depleting systemic exosomes from the circulation of cancer patients and may de-repress immunological functions thereby allowing anti-tumor responses.

### EXAMPLES

### Example 1

### Selective Protein Removal with Antibodies

The utility of the HEMOPURIFIER® affinity capture device for the specific removal of proteins through affinity hemofiltration has been demonstrated. Selective protein binding was demonstrated by investigating the efficacy of human immunodeficiency virus (HIV) gp120 removal using acellular fluids such as tissue culture media and PBS. The HL2/3 cell line used (AIDS Resource and Program, Rockville, Md.) contained a replication deficient, noninfectious virus secreting the envelope protein HIV gp120 into its culture media. Culture supernatant was continually recirculated over the anti-gp120 antibody affinity HEMOPURIFIER® affinity capture device for 6 hours.

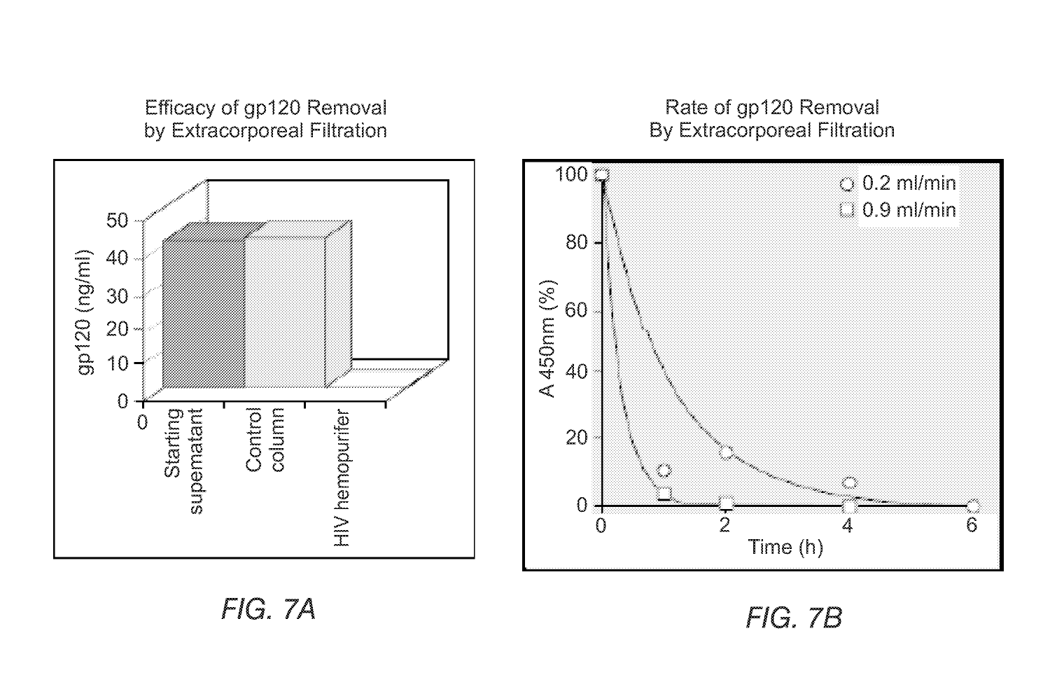

As seen in FIG. 7A almost complete removal of gp120 was achieved as measured by ELISA. This suggests the potency of the HEMOPURIFIER® affinity capture device to selectively remove proteins since the control column without anti-gp120 antibodies failed to remove gp120. In order to establish the rate of removal of gp120 in buffer using affinity dialysis and to test whether a faster flow rate would cause more rapid adsorption of gp120 to the immobilized substrates, the flow through rate was varied. HIVgp120 at a concentration of 100 ng/ml in PBS was circulated over the HEMOPURIFIER® affinity capture device containing goat anti-HIV IgG (2.1 mg/ml) covalently coupled to 1% agarose similarly to the above experiment. Two different flow rates were used: 0.2 ml/min and 0.9 ml/min, at room temperature. As seen in FIG. 7B, the higher flow rate allowed a more rapid removal of gp120.

### Example 2

### Selective Virus Removal with Lectins

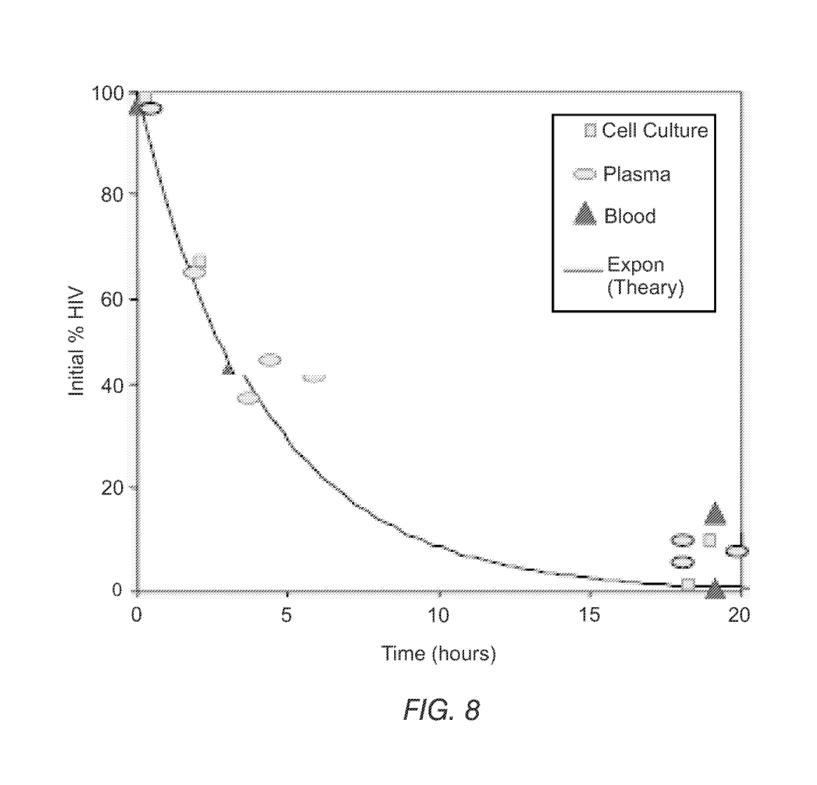

The size of the HIV virus (˜100 nm) is comparable to the size of exosomes. Based upon success of selectively depleting proteins in the acellular media, the next set of experiments involved depletion of virus from whole blood. These experiments demonstrate the use of the HEMOPURIFIER® affinity capture device as an effective means for removal of HIV with the plant lectin Galanthus Nivalis Agglutinin (GNA) and as a model system for the removal of any monovalent or multivalent glycoprotein, glycoprotein coated exosomes or other biomarkers (FIG. 8).

The in vitro affinity cartridge used in the experiments was a 0.5×10 cm long MicroKros polyethersulfone hollow-fiber dialysis cartridge (0.5 ml internal volume, hollow fibers 200 m ID×240 m OD, pore diameter<200 nm) equipped with Luer fittings. FIG. 8 shows that 100 nm glycoprotein coated particles (HIV) can be removed from culture fluids, plasma and infected blood using an antibody-based affinity hemofiltration system. Briefly, a volume of 15 ml of HIV-1 infected cell culture fluids, plasma, or blood was pumped through the cartridge using a peristaltic pump at a flow rate of 0.9 ml/min. Typical virus levels before exposure to the device in blood, plasma or cell culture supernatants were around 1-2.3×10⁵ viral copies per ml with the highest loads in blood. At various intervals, small samples were taken and virus measured by quantitative PCR and p24 ELISA. Removal follows an apparent first order path (t_{1/2}˜2.8 h) regardless of the carrier fluid, the result expected for antibody-antigen reactions when antibody is in excess. Of interest is the apparent binding capacity of the cartridge. This module could contain 10 mg of GNA lectin, and theoretically remove over 10¹⁵ virus particles or up to 100,000 times the average daily production of HIV. These data demonstrate the effectiveness of the HEMOPURIFIER® affinity capture device to remove glycoprotein coated particles of the same size and nature as HIV, tumor derived exosomes or other biomarkers.

In further experiments, HIV bound to the affinity cartridge was eluted by extracting the entire cartridge with TRI-LS reagent to extract viral RNA. In these experiments, the viral RNA removed from the cartridge appeared to contain all of the RNA removed from the culture fluid. Thus it was shown that viral RNA could be nearly quantitatively recovered from the cartridge in concentrated form.

Further, the capture of hepatitis virus C (HCV) from the blood of intermittent dialysis patients co-infected with HCV (Table 1) also demonstrates the capacity of the device to isolate, concentrate and remove a glycoprotein coated particle of ˜50 nm (HCV) from patients with high circulating concentrations of (virus) particles. In clinical studies presented in Example 4, HCV RNA was also recovered from Hemopurifier cartridges in concentrated form.

### Example 3

### Exosome Removal

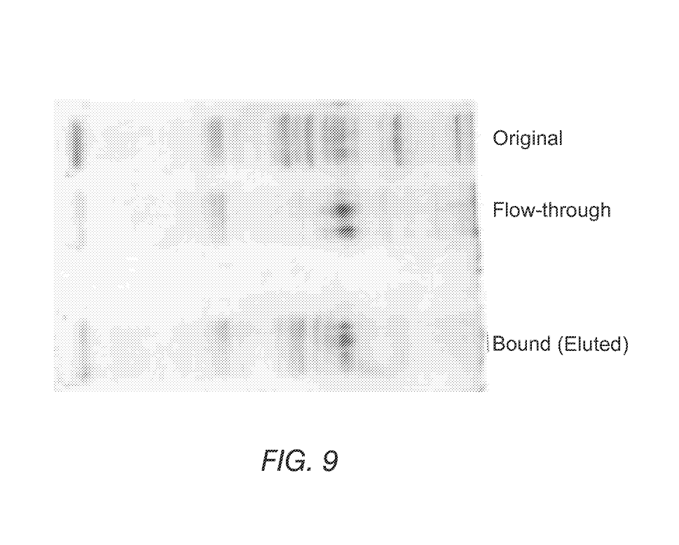

To demonstrate the utility of affinity cartridges to specifically isolate tumor exosomes, chromatographically isolated exosomes from ovarian cancer patients were applied to the cartridges in 10 ml TBS. An aliquot of this material was retained for electrophoretic analysis, and the results of the analysis are illustrated in FIG. 9.

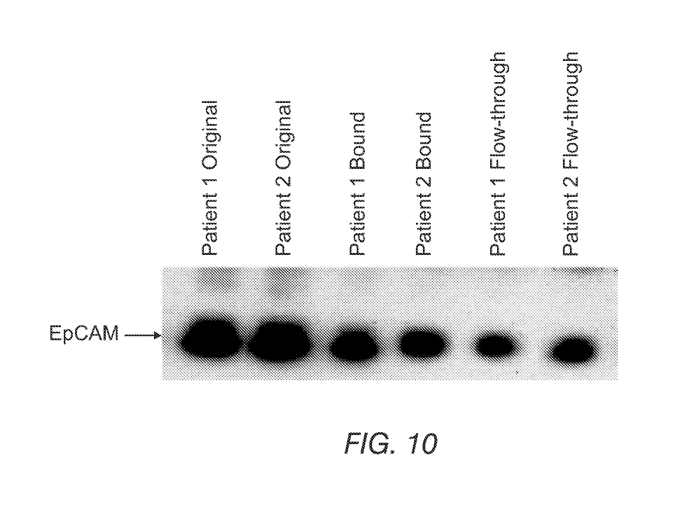

The material that did not bind on this initial pass-through the cartridge was collected and retained for electrophoretic analysis. The bound exosomes were eluted from the cartridges in 10 ml of 1× Laemmli sample buffer. These materials were then separated on an 8% polyacrylamide SDS gels and the components visualized by colloidal blue staining. This material was further examined for the expression of the tumor associated exosomal marker, EpCAM, by western immunoblotting (FIG. 10).

These results demonstrated the specific binding of exosomal material to the HEMOPURIFIER® affinity capture GNA cartridges. A single pass through the HEMOPURIFIER® affinity capture cartridge resulted in a 60.7±5.3% reduction in the level of tumor exosomes.

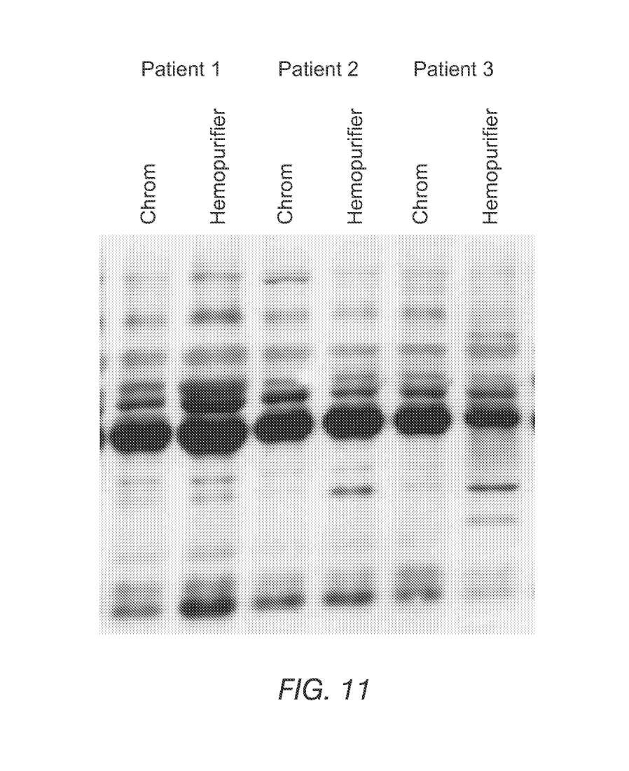

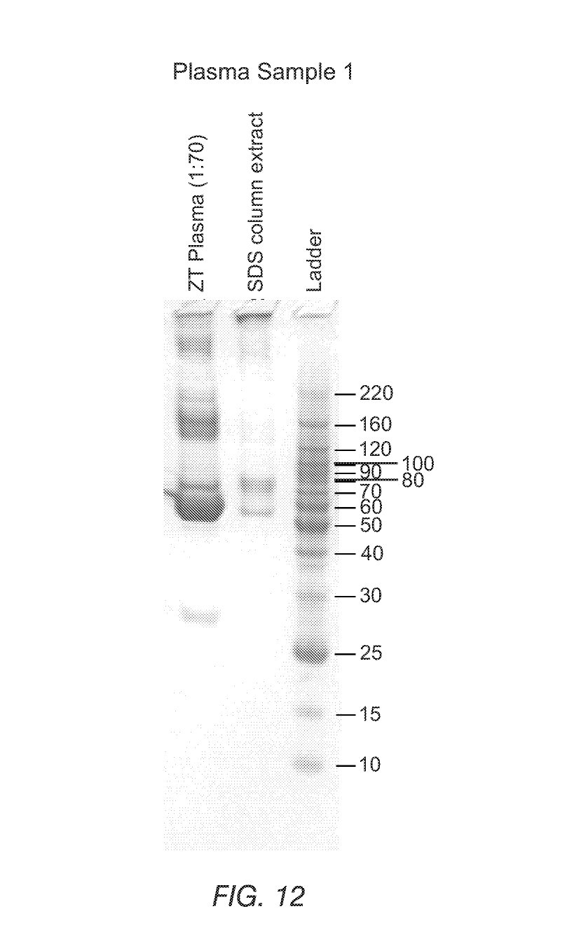

To define the ability of cartridges to isolate exosomes from biologic fluids, unfractionated ascites were applied to the cartridges and the bound material eluted. This eluted material was compared to chromatographically isolated exosomes from the same ascites (FIG. 11). The material isolated by both approaches appears to be identical.

### Example 4

### Clinical Safety Data

A phase I clinical trial was conducted to demonstrate safety of the Aethlon HEMOPURIFIER® affinity capture device in a clinical setting. Twenty-four HEMOPURIFIER® affinity capture cartridges packed with GNA lectin were tested on four study subjects having Hepatitis C virus (HCV) infected end stage renal disease (ESRD). Each cartridge purified the blood of study subjects for up to four hours in six sequential treatments per patient every two to three days. Table 1 summarizes the results for capture and isolation of Hepatitis C Virus from HCV+ patients undergoing intermittent dialysis and treatment with the HEMOPURIFIER® affinity capture device. These results clearly show that HCV viral RNA was concentrated on the cartridge and could be recovered in high yield.

TABLE 1

| | | Virus capture in | | | Initial total plasma | | |
|---|---|---|---|---|---|---|---|
| | | HEMOPURIFIER | | | viral load | | |
| | | Number | Average copies of | ±2 | Virus | ±2 | %/HCV |
| Patient | Cartridge | of tests | HCV virus/cartridge | SEM | (copies) | SEM | captured |
| 1 | H6 | 6 | 1,986,500,944 | 8.4% | 3,803,850,177 | 17% | 52% |
| 2 | H1 | 3 | 225,709,477 | 35% | 2,486,414,328 | 10% | 9% |
| | H6 | 3 | 119,015,261 | | | | 5% |
| 3 | H1 | 3 | 515,201,540 | 41% | 1,602,209,244 | 22% | 32% |
| | H6 | 3 | 501,604,531 | | | | 31% |
| 4 | N/A | | N/A | | N/A | | N/A |
| Average | | | 889,088,783 | | 2,630,824,583 | | 30% |

N/A: not available

Treatment with HEMOPURIFIER® affinity capture device was considered safe and well tolerated in this study with only two adverse effects observed in a single treatment, of one patient, those being mild nausea and severe shivering, events that might be occasionally anticipated from intermittent dialysis procedures.

### Example 5

### Devices

Construction of Affinity Chromatography Cartridges.

To test the effectiveness of various matrix formulations, affinity matrices are constructed using several different antibodies and lectins directed toward the capture of known circulating cancer biomarkers. Prostate cancer biomarkers that are tested include PSA, Cathepsin D, hK2 and AMACR.

In another embodiment, antibodies and lectins directed toward the capture of tumor-derived exosomes are used. These cartridges are used to isolate and concentrate exosomes from cancer cell supernatants and serum samples spiked with known quantities of exosomes. Studies are conducted to establish the efficiency and selectivity of affinity cartridges to quantitatively remove exosomes from complex fluid mixtures, and to standardize methods for optimal elution, detection, and quantification of exosomes specifically bound to the affinity cartridges. These methods are then used for selective depletion of exosomes from ovarian cancer patient sera.

Antibody- and lectin-coupled affinity substrates are constructed. Antibody affinity has several advantages including high specificity and high avidity. Antibodies against PSA, cathepsin D, hK2 and AMACR are readily available. Antibodies against markers of tumor-derived exosomes are readily available. Lectin affinity chromatography has additional benefits over antibodies. Lectins are much more resistant to degradation due to proteolysis and are capable of withstanding greater variations in acidity and temperature than antibodies. In addition, lectins are smaller than antibodies thereby allowing a higher density of affinity reagent on the matrix. Finally, lectins may prove to be useful in isolating previously uncharacterized glycoproteins shed into the serum. Both of these classes of affinity matrices are first tested using traditional chromatography cartridges to ensure that the substrates provide adequate sensitivity and specificity. The HEMOPURIFIER® affinity capture cartridges are then constructed using these affinity substrates and are used for isolation of the cancer biomarkers from cell cultures and sera.

Production of Lectin-Coupled Substrate.

Synthesis cartridges are prepared containing affinity resin to assess the efficacy of three lectins by affinity hemofiltration to capture, concentrate and selectively remove exosomes from tissue culture supernatants and human sera. Three lectins are used: Concanavalin A (ConA), Galanthus nivalis agglutinin (GNA), and cyanovirin (CVN). These lectins are commercially available from Avecia (Milford, Mass.) and Sigma (St. Louis, Mo.). The lectins are covalently coupled to an amino-Celite substrate. Amino-Celite (Chromasorb GAW60/80, Celite Corp. Lompoc, Calif.) is prepared by overnight reaction of the Celite (silicate containing diatomaceous earth) in a 5% aqueous solution of γ-aminopropyl triethoxysilane. The aminated Celite is washed free of excess reagent with water and ethanol and dried overnight to yield an off-white powder. The powder is then suspended in 5% glutaraldehyde (Sigma, St. Louis, Mo.) for 30 minutes. Excess glutaraldehyde is removed by standard filtration and washed with water until no detectable aldehyde remains in the wash using Schiff's reagent as an indicator. The filter cake is resuspended in cyanoborohydride coupling buffer (Sigma, St. Louis, Mo.) containing the lectin and the reaction is allowed to proceed overnight at room temperature. At the end of the reaction, unreacted lectin is washed off and the unreacted aldehyde is aminated with ethanolamine. After final washings with 5-10 column volumes of sterile PBS, the material is stored at 4° C. until use. Up to 4 kg of this lectin resin can be produced in bulk and employed for lectin affinity chromatography using either open system filter columns for quality control testing of tissue culture supernatant or plasma, or for final use within the HEMOPURIFIER® affinity capture device for the testing of blood.

Production of Antibody-Coupled Substrate.

A hemofiltration cartridge with a Sepharose matrix covalently attached to anti-gp120 antibodies or GNA can selectively remove gp120 envelope protein and HIV virions (FIG. 7A, FIG. 7B and FIG. 8) (Tullis, R. H., et al., Affinity hemodialysis for antiviral therapy. I. Removal of HIV-1 from cell culture supernatants, plasma, and blood. Ther Apher, 2002. 6(3): 213-20; and Tullis, R. H., et al., Affinity hemodialysis for antiviral therapy. II. Removal of HIV-1 viral proteins from cell culture supernatants and whole blood. Blood Purif, 2003. 21(1):58-63).

In one embodiment affinity substrates are prepared to selectively remove prostate cancer biomarkers with antibodies. Anti-cathepsin D, anti-PSA, and anti-hK2 antibodies are purchased from Santa Cruz Biotechnology (Santa Cruz, Calif.). Anti-AMACR antibodies are purchased from Chemicon (Temecula, Calif.). A variety of antibodies to biomarkers that have been shown to be correlated with outcome are characterized. These biomarkers include sFRP1, 14-3-3 zeta and delta, Sparc 1, and others. These biomarkers exhibit differential expression in prostate cancer based upon western blot, IP, and TMA studies. These antibodies therefore provide a source of anti-biomarker antibodies for cartridge construction.

In another embodiment, affinity substrates are prepared to selectively remove cancer exosomes, which are similar in size to HIV virions, with antibodies directed at the two surface antigens PLAP and FasL. The anti-PLAP and anti-FasL antibodies are purchased from Pharmingen (San Diego, Calif.).

In another embodiment, affinity substrates are prepared to use antibodies to affinity capture cathepsin D and galectin-3. Anti-cathepsin D and anti-galectin-3 antibodies are purchased from Santa Cruz Biotechnology (Santa Cruz, Calif.). Antibody-Celite matrix is produced as described above for lectin conjugations.

Production of Affinity-Coupled Hemopurifier® Cartridges.

The lectin and antibody resins demonstrating the most efficient removal of cathepsin D, PSA, hK2 and AMACR (as well as novel biomarkers), Galectin-3, and tumor ovarian tumor exosomes in the experiments described below (in Example 6) are used to build affinity cartridges, preferably HEMOPURIFIER® affinity capture cartridges. Cartridges with the resin only are prepared for control studies.

Briefly, the HEMOPURIFIER® affinity capture device is made using a suspension of the affinity matrix in buffer (typically PBS) and pumping the slurry into the extra-fiber spaces of a hollow-fiber plasmapheresis cartridge with a peristaltic pump. Filling is performed under sterile conditions. To prevent overpacking, it is preferable to keep pump pressures below 100 psi. A Medica plasmapheresis cartridge (Medollo, Italy) equipped with Luer fittings is used. The hollow-fiber membranes in this device have an average pore size of 200 nm. Cartridges may be stored in the refrigerator prior to quality control testing or packaged and sterilized by irradiation. Quality control testing procedures include fiber and cartridge integrity, complement activation, pyrogen and sterility testing, accelerated stability and leaching, and protein binding capacity.

### Example 6

### Characterization and Optimization of Biomarker Capture Using Affinity Chromatography Cartridges—Prostate Biomarkers Ovarian Cancer Biomarkers and Removal of Ovarian Cancer Exosomes

Affinity cartridges, preferably HEMOPURIFIER® affinity capture cartridges, are used to isolate cancer biomarkers from cancer cell supernatants and serum samples spiked with known quantities of biomarkers. Studies are conducted to establish the efficiency and selectivity of affinity cartridges to quantitatively remove selected biomarkers from complex fluid mixtures, and to standardize methods for optimal elution, detection, and quantification of enriched cancer biomarkers specifically bound to the cartridges.

Studies are conducted to establish which of the lectin- and antibody-affinity formulations constructed in Example 5 are best suited for the isolation of prostate and ovarian cancer biomarkers Cathepsin D, PSA, hK2 and AMACR, galectin-3, (as well as our novel biomarkers) and tumor-derived exosomes from clinical serum samples. The ability of the lectin- and antibody-affinity matrices to quantitatively remove known quantities of spiked biomarkers from liquids of increasing complexity: a) PBS, b) Tissue-culture growth media, c) Heparanized blood is tested. The binding and elution conditions are optimized during these experiments to maximize binding while maintaining specificity.

Once conditions are optimized using known amounts of biomarkers, the affinity cartridges are used to isolate and concentrate cancer biomarkers from tissue-culture supernatants derived from exponentially growing prostate cancer cell lines, PC3 and LNCaP, since they are well-characterized prostate tumor cell line known to produce detectable levels of markers (Vaisanen, V., et al., Development of sensitive immunoassays for free and total human glandular kallikrein 2. Clin Chem, 2004. 50(9):1607-17; Bindukumar, B., et al., Prostate-Specific Antigen Modulates the Expression of Genes Involved in Prostate Tumor Growth. Neoplasia, 2005. 7(5):544, incorporated by reference in their entireties). Alternatively, other prostate cancer cell lines (DU145, PwR-1E, MDA PCA-2b, LAPC-4) or primary cultures of tumor-derived human prostate tissue obtained from radical prostatectomy are used if PC3 or DU145 cells fail to secrete sufficient levels of prostate cancer biomarkers for this study.

The affinity cartridges are used to isolate and concentrate cancer biomarkers (Cathepsin-D, galectin-3), and tumor-derived exosomes from tissue-culture supernatants derived from exponentially growing ovarian cancer cell lines. OVCAR-3 ovarian tumor cells are used since they are a well characterized ovarian tumor cell line known to contain detectable levels of exosomes. Alternatively, other ovarian cancer cell lines (Dov13, OvMz, TOV-112D, SKOV-3) are used if OVCAR-3 cells fail to secrete sufficient levels of ovarian cancer biomarkers (Cathepsin-D, galectin-3, and tumor-derived exosomes) for this study.

### Isolation of Prostate Cancer Biomarkers for Spiking Experiments

Culture of PC-3 and LNCaP Cells.

Prostate tumor cell lines (ATTC, Rockville, Md.) are grown in RPMI medium supplemented with 10% fetal bovine serum, 0.1 mM nonessential amino acids, 1 mM sodium pyruvate, 200 mM l-glutamine, 100 ng/ml streptomycin, and 100 IU/ml penicillin in a humidified 5% CO₂ incubator. Conditioned media from exponentially growing sub-confluent (80-90% confluent) cultures is used for isolation of cathepsin-D, PSA, hK2, and AMACR.

Source of proteins for spiking experiments. Purified cathepsin D isolated from human spleen (Calbiochem) is used for spiking of tissue culture supernatant and blood samples. PSA isolated from human seminal fluid (Calbiochem), recombinant human AMACR and human kallikrein 2 (Abnova) is used for spiking of tissue culture supernatant and blood samples.

### Quantification and Characterization of Prostate Cancer Biomarkers

Quantification of PSA.

Levels of PSA (Total PSA, free PSA, and alpha-chymotrypsin-complexed PSA) in unfractionated and fractionated tissue culture and blood samples are determined using PSA ELISA kits (Alpha diagnostics) as per manufacturer's instructions.

Quantification of Cathepsin D.

Levels of cathepsin D in unfractionated and fractionated tissue culture and blood samples are determined using a standard cathepsin D ELISA kit (Calbiochem) as per manufacturer's instructions.

Quantification of hK2.

Levels of hK2 in unfractionated and fractionated tissue culture and blood samples are determined using a standard hK2 ELISA kit (Hybritech; Beckman Coulter) as per manufacturer's instructions. This assay measures both free and hK2 complexes and has been shown to have minimal cross-reactivity with PSA.

Quantification of AMACR.

Levels of hK2 in unfractionated and fractionated tissue culture and blood samples are determined using ELISA. Briefly, 96 well plates are coated with 100 μl of 2.5 μg/ml AMACR mAb (Santa Cruz Biotechnology) and incubated overnight at 4° C. After blocking with 0.1% Tween-20 in 1×TBS, 100 μl of tissue culture or blood samples containing AMACR at concentrations between 0.1 and 100 ng/ml in TBS/0.1% BSA is added to the antibody coated wells and incubated for 2 hours at 4° C. Wells are washed with 0.1% Tween-20 in 1×TBS and incubated with a HRP-conjugated anti-AMACR detection antibody conjugate followed by ABTS detection. Standard curves are generated using samples spiked with known amounts of AMACR recombinant proteins.

Quantification of Novel Prostate Biomarkers.

Quantification of other markers including sFRP1, 14-3-3 zeta and delta, Sparc 1 will be done via western analysis.

### Optimization of Biomarker Capture Using Affinity Chromatography Cartridges

Selective Removal of Cathepsin D Using Affinity Chromatography Cartridges

One ml lectin-Celite columns with GNA, ConA, MAA or anti-cathepsin D, or unconjugated Celite to control for nonspecific binding are used. Twenty ml of PBS spiked with varying amounts of cathepsin D (1 ng/ml-1 μg/ml) are circulated 3-5 times over each of the affinity-Celite columns at room temperature. The circulating samples are tested after each pass with respect to cathepsin D using cathepsin D ELISA, Cathepsin D is eluted from the matrix and quantified by ELISA. These spiking experiments are repeated using more complex mixtures. These include tissue culture media used for culture of PC3 and LNCaP tumor cells and heparinized blood from healthy donors. Non-specific binding of proteins from these mixtures is assessed by SDS-page gel electrophoresis. Washing conditions of increasing stringency are applied to ensure maximum cathepsin D binding and minimal non-specific binding of other serum proteins. Affinity matrices demonstrating the highest sensitivity and specificity are used for construction of affinity chromatography cartridges. Similar spiking experiments are conducted using the cartridges. Briefly, twenty ml of heparinized blood spiked with varying amounts of cathepsin D (1 ng/ml-1 μg/ml) are run through the extracorporeal system with the lectin or antibody resin or the resin alone at a flow rate of 0.6-0.9 ml per minute for up to 5 hours. After each pass the circuit is stopped and a small aliquot of the circulating fluid will be removed. Amounts of bound and unbound cathepsin D are determined by ELISA.

Selective Removal of PSA Using Affinity Chromatography Cartridges.

One ml lectin-celite columns with GNA, ConA, MAA or anti-PSA, or unconjugated celite to control for unspecific binding are used. Spiking experiments are conducted as described for cathepsin D with the use of PSA ELISA for quantification.

Selective Removal of hK2 Using Affinity Chromatography Cartridges.

One ml lectin-celite columns with GNA, ConA, MAA or anti-hK2, or unconjugated celite to control for unspecific binding are used. Spiking experiments are conducted as described for cathepsin D with the use of hK2 ELISA for quantification.

Selective Removal of AMACR Using Affinity Chromatography Cartridges.

One ml lectin-Celite columns with GNA, ConA, MAA or anti-AMACR, or unconjugated Celite to control for unspecific binding are used. Spiking experiments are conducted as described for cathepsin D with the use of AMACR ELISA for quantification.

Optimization of Biomarker Capture from Prostate Cancer Cell Supernatants Using Affinity Chromatography Cartridges

Removal of Cathepsin D, PSA, hK2, and AMACR from Prostate Cancer Tissue Culture Supernatants.

The optimal affinity chromatography cartridges and conditions established in the previous experiments are used to isolate these potential biomarkers from PC3 and LnCaP tissue culture supernatants. Prostate cancer cells are grown as described above and aliquots of tissue culture supernatants applied to the affinity chromatography cartridges, preferably HEMOPURIFIER® affinity capture cartridges, as described above. The bound and unbound fractions are characterized by ELISA.

### Isolation of Ovarian Cancer Biomarkers for Spiking Experiments

Culture of OVCAR-3 Cells.

The OVCAR-3 ovarian tumor cell line (ATTC, Rockville, Md.) is grown in Dulbecco's modified Eagles medium supplemented with 10% fetal bovine serum, 0.1 mM nonessential amino acids, 1 mM sodium pyruvate, 200 mM 1-glutamine, 100 ng/ml streptomycin, and 100 IU/ml penicillin in a humidified 5% CO₂ incubator. Conditioned media from exponentially growing sub-confluent (80-90% confluent) cultures is used for isolation of galectin-3, cathepsin D, and exosomes.

Source of Galectin-3 and Cathepsin D for Spiking Experiments.

Purified cathepsin D isolated from human spleen (Calbiochem) will be used for spiking of tissue culture supernatant and blood samples. Human recombinant galectin-3 (Calbiochem) will be used for spiking of tissue culture supernatant and blood samples.

Exosome Isolation from Blood and OVCAR-3 Cultures.

For spiking experiments, exosomes are purified from OVCAR-3 ovarian tumor cell supernatants or heparinized blood of healthy volunteers and cancer patients according to described methods (Taylor, D. D., S. Akyol, and C. Gercel-Taylor, Pregnancy-associated exosomes and their modulation of T cell signaling. J Immunol, 2006. 176(3):1534-42; Taylor, D. D., K S. Lyons, and C. Gercel-Taylor, Shed membrane fragment-associated markers for endometrial and ovarian cancers. Gynecol Oncol, 2002. 84(3):443-8; Taylor, D. D., et al., Modulation of CD3-zeta as a marker of clinical response to IL-2 therapy in ovarian cancer patients. Gynecol Oncol, 2004. 94(1):54-60). Briefly, heparinized plasma is purified from 10-30 ml of peripheral blood by centrifugation at 500×g for one half-hour. Separation of cellular debris is accomplished by a second centrifugation at 7,000×g for an additional half-hour. Exosomes are subsequently be collected by centrifugation at 100,000×g for 3 hours, followed by a washing step in PBS under the same conditions. Using this procedure, approximately 0.5 μg/ml or <250 μg/ml of exosomal protein is isolated from healthy volunteers as determined by the Bradford Assay (Bio-Rad, Hercules, Calif.). The plasma of ovarian cancer patients typically generates a higher exosomal yield, ranging between 1,100 to 2,500 μg/ml. This is in agreement with studies describing high concentrations of circulating “membrane vesicles” found systemically in cancer patients.

### Quantification and Characterization of Ovarian Cancer Biomarkers

Characterization of Isolated Ovarian Cancer Exosomes.

Concentration of surface markers MHC I, PLAP, and FasL is assessed on exosomes from healthy volunteers and cancer patients using the microbead method and/or a direct flow cytometric method. In the microbead method, beads coated with anti-CD63 capture CD63-expressing exosomes, which then can be labeled with antibodies directed at different exosomal surface antigens following FACS analysis. Briefly, 10 μl of 4-μm-diameter aldehyde/sulfate latex beads (Interfacial Dynamics, Portland, Oreg., USA) are incubated with purified anti-CD63 mAb at room temperature in a small volume (50 μl). After 15 min, the volume is adjusted to 400 μl with PBS and incubated overnight at 4° C. under gentle agitation; the reaction is then stopped by incubation for 30 min in PBS supplemented with 100 mM glycine. Exosomes are incubated for 15 min at 4° C. with the anti-CD63-latex beads. The volume is subsequently brought to 400 μl with PBS and incubated for 2 h at 4° C. Microvesicles-coated beads are washed twice in FACS washing buffer (1% BSA and 0.1% NaN3 in PBS) by centrifugation at 500×g for 15 minutes and re-suspended in 400 μl FACS washing buffer, stained with fluorescent antibodies and analyzed on a FACSCalibur flow cytometer (BD Biosciences) and CellQuest software. Fluorescent antibodies to MHC I, PLAP, and FasL are purchased from Immunotech (Westbrook, Me.) and BD Pharmingen (San Diego, Calif.). Labeling of exosomes with anti-FasL, anti-PLAP, and anti-MHC I antibodies alone as well as in combination using double and triple labeling procedures will determine which reagents can best distinguish between cancer exosomes and those from healthy volunteers.

Quantification of Galectin-3.

Levels of galectin-3 in unfractionated and fractionated tissue culture and blood samples are determined using a standard galectin-3 ELISA kit (Calbiochem) according to the manufacturer's instructions.

Quantification of Cathepsin D.

Levels of Cathepsin D in unfractionated and fractionated tissue culture and blood samples will be determined using a standard cathepsin D ELISA kit (Calbiochem) as per manufacturer's instructions.

### Optimization of Biomarker Capture Using Affinity Chromatography Cartridges

Selective Removal of Cathepsin D Using Affinity Chromatography cartridges.

One ml lectin-celite columns with GNA, ConA, CVN or anti-cathepsin D, or unconjugated celite to control for nonspecific binding are used. Twenty mls of PBS spiked with varying amounts of cathepsin D (1 ng/ml-1 μg/ml) are circulated 3-5 times over each of the affinity-celite columns at room temperature. The circulating samples are tested after each pass with respect to cathepsin D using cathepsin D ELISA, Cathepsin D is eluted from the matrix and quantified by ELISA. These spiking experiments are then repeated using more complex mixtures. These include tissue culture media used for culture of OVCAR-3 ovarian tumor cells and heparinized blood from healthy donors. Non-specific binding of proteins from these mixtures is assessed by SDS-page gel electrophoresis. Washing conditions of increasing stringency is applied to ensure maximum cathepsin D binding and minimal nonspecific binding of other serum proteins. Affinity matrices demonstrating the highest sensitivity and specificity are used for construction of affinity chromatography cartridges, preferably HEMOPURIFIER® affinity capture cartridges. Similar spiking experiments are conducted using the cartridges. Briefly, twenty ml of heparinized blood spiked with varying amounts of cathepsin D (1 ng/ml-1 μg/ml) are run through the extracorporeal system with the lectin or antibody resin or the resin alone at a flow rate of 0.6-0.9 ml per minute for up to 5 hours. After each pass the circuit is stopped and a small aliquot of the circulating fluid is removed. Amounts of bound and unbound cathepsin D will be determined by ELISA.

Selective Removal of Galectin-3 Using Affinity Chromatography Cartridges.

One ml lectin-celite columns with GNA, ConA, CVN or anti-galectin-3, or unconjugated celite to control for unspecific binding are used. Spiking experiments are conducted as described for cathepsin D with the use of galectin-3 ELISA for quantification.

Selective Removal of Exosomes Using Affinity Chromatography Cartridges.

One ml lectin-Celite columns with either GNA, ConA, CVN, anti-PLAP, anti-FasL, or unconjugated Celite to control for unspecific binding are used. Spiking experiments are conducted as described for cathepsin D using exosomes isolated from ovarian cancer patient sera or OVCAR-3 cultures at concentrations between 1-1000 μg/ml. Concentrations of bound and unbound exosomes will be assessed as described above.

Optimization of Biomarker Capture from Ovarian Cancer Cell Supernatants Using Affinity Chromatography Cartridges

Removal of Cathepsin D, Galectin-3, and Tumor Exosomes from OVAR-3 Tissue Culture Supernatants.

The optimal affinity chromatography cartridges and conditions established in the previous experiments are used to isolate these potential biomarkers from OVAR-3 tissue culture supernatants. OVAR-3 cells are grown as described and aliquots of tissue culture supernatants applied to the affinity chromatography cartridges as described above. The bound and unbound fractions are characterized by ELISA.

### Optimization of Exosome Capture Using Affinity Chromatography Cartridges

Selective Removal of Exosomes Using Affinity Chromatography Cartridges

One ml lectin-Celite columns with either GNA, ConA, CVN, anti-PLAP, anti-FasL, or unconjugated Celite to control for unspecific binding are used. Spiking experiments are conducted using exosomes isolated from ovarian cancer patient sera or OVCAR-3 cultures at concentrations between 1-1000 μg/ml. Concentrations of bound and unbound exosomes are assessed as described above. For spiking experiments 20 ml of PBS spiked with varying amounts of tumor exosomes (1-1000 μg/ml) are circulated 3-5 times over each of the affinity-celite columns at room temperature. The circulating samples are tested after each pass with respect to exosomes composition. Bound material is eluted from the matrix and exosomes characterized in a similar fashion. These spiking experiments are then repeated using more complex mixtures. These include tissue culture media used for culture of OVCAR-3 ovarian tumor cells and heparinized blood from healthy donors. Non-specific binding of proteins from these mixtures is assessed by SDS-page gel electrophoresis. Washing conditions of increasing stringency is applied to ensure maximum exosome binding and minimal non-specific binding of other serum proteins. Affinity matrices demonstrating the highest sensitivity and specificity are used for construction of affinity chromatography cartridges, preferably HEMOPURIFIER® affinity capture cartridges. Similar spiking experiments are conducted using the cartridges. Briefly, twenty ml of heparinized blood spiked with varying amounts of exosomes (1-1000 μg/ml) is run through the extracorporeal system with the lectin or antibody resin or the resin alone at a flow rate of 0.6-0.9 ml per minute for up to 5 hours. After each pass the circuit is stopped and a small aliquot of the circulating fluid is removed. Amounts of bound and unbound exosomes are determined by microbead assay or FACS.

### Example 7

### Biomarker and Exosome Capture from Clinical Samples Using Affinity Chromatography Cartridges—Prostate Biomarkers, Ovarian Cancer Biomarkers, and Removal of Ovarian Cancer Exosomes

### Biomarker Capture Using Affinity Chromatography Cartridges—Prostate Cancer

Affinity cartridges, preferably HEMOPURIFIER® affinity capture cartridges, are used to isolate cancer biomarkers from serum of healthy volunteers and patients diagnosed with BPH or different stages of prostate cancer using standardized methods developed in Example 6. Relative sensitivity and enrichment of circulating biomarkers using the cartridges are compared to standard ELISA analysis of serum samples.

Serum Samples.

Blood samples from BPH, prostate cancer patients (stage I-IV) and healthy controls are obtained. A bank of prostate cancer tissues and fluids based on a clinical observational study of over 900 consented patients is used. The tissues have been studied extensively by expression analysis and predictive biomarkers have been identified. Recently, this study has been extended to banking serum plasma, and postDRE urine specimens and these are available for this study.

Affinity Chromatography Cartridge Capture of Cathepsin D from Blood.

Cathepsin D is isolated from normal serum and sera collected from BPH and prostate cancer patients using the optimal affinity chromatography cartridge formulation and procedures defined in Example 6. Standard cathepsin D ELISA determines the levels of cathepsin D in the blood samples prior to affinity separation by the affinity chromatography cartridge. Cathepsin D levels of bound and unbound fractions are assessed.

Affinity Chromatography Cartridge Capture of PSA from Blood.

PSA is isolated from normal serum and sera collected from BPH and prostate cancer patients using the optimal affinity chromatography cartridge formulation and procedures established in Example 6. Standard PSA ELISA is used to determine the levels of free and complexed PSA in the blood samples prior to affinity separation by the affinity chromatography cartridge. Free and complexed PSA levels of bound and unbound fractions are assessed.

Affinity Chromatography Cartridge Capture of hK2 from Blood.

hK2 is isolated from normal serum and sera collected from BPH and prostate cancer patients using the optimal affinity chromatography cartridge formulation and procedures established in Example 6. hK2 ELISA determines the levels of hK2 in the blood samples prior to affinity separation by the affinity chromatography cartridge. hK2 levels of bound and unbound fractions are assessed.

Affinity Chromatography Cartridge Capture of AMACR from Blood.

AMACR is isolated from normal serum and sera collected from BPH and prostate cancer patients using the optimal affinity chromatography cartridge formulation and procedures established in Example 6. ELISA determines the levels of AMACR in the blood samples prior to affinity separation by the affinity chromatography cartridge. AMACR levels of bound and unbound fractions are assessed.

Pathological Staging and In Vitro Immunohistochemistry.

For selective cases where paired tissue samples are available, prostate tissue samples from serum donors are analyzed in order to correlate quantitative measurements of serum biomarkers obtained in these studies with clinical stage and Gleason score of the tissue biopsy. In addition, immunohistochemical distribution of the prostate cancer biomarkers in tissue biopsies is determined. The tissue samples to be examined here are routinely collected in the operating room and specimens are immediately transported to institutional pathologists. Both tumor and non-tumor tissue are isolated. A portion of the specimen to be analyzed is removed for histological analysis. The remaining portion is cut into small pieces (1-3 mm²) and submerged in ViaSpan organ preservation solution. Tissue explants are immediately plated into 6-well tissue culture dishes coated with type I collagen (InVitrogen) in PrEGM growth media (Clonetics) for primary culture. The remaining tissue explants are cryopreserved using 10% DMSO with 90% PrEGM media for future use. Tissues identified by the pathologist to be of the appropriate Gleason score are used for subsequent analysis. Paraffin sections are prepared and indirect immunohistochemistry is performed using antibodies against the serum markers used in this study. Cell-type specificity of prostate biomarkers is determined by staining tissue sections with antibodies directed against pan-cytokeratin (Sigma) to identify epithelial cells and antibodies against alpha-smooth muscle actin and prolyl-4-hydroxylase to identify stromal cells. Other markers are available that will permit further differentiation of the epithelial cells into luminal, basal, and neuroendocrine cells.

More Affinity Reagents.

The lectins selected for initial studies were chosen because they are known to bind some characterized prostate cancer biomarkers and were successfully used in the construction of affinity chromatography HEMOPURIFIER® affinity capture cartridges for the biocapture of virus proteins and viral particles.

More Prostate Cancer Biomarkers.

The affinity chromatography cartridges are validated using four prostate cancer markers (PSA, hK2, cathepsin D, and AMACR) because these markers are detectable in the blood and have clinical significance. If any of these markers fail to meet specific criteria, alternative prostate cancer biomarkers will be selected.

### Biomarker and Exosome Capture Using Affinity Chromatography Cartridges—Ovarian Cancer

Affinity cartridges, preferably HEMOPURIFIER® affinity capture cartridges, are used to isolate cancer biomarkers from serum of normal volunteers and patients diagnosed with different stages of ovarian cancer using standardized methods developed in Example 6. Relative sensitivity and enrichment of circulating biomarkers using the affinity cartridges are compared to standard ELISA analysis of serum samples.

Serum Samples.

Blood samples from ovarian cancer patients (stage 1-IV) and healthy controls are obtained. All specimens are obtained under an informed consent protocol.

Affinity Chromatography Cartridge Capture of Cathepsin D from Blood.

Cathepsin D is isolated from normal serum and sera collected from ovarian cancer patients using the optimal affinity chromatography cartridge formulation and procedures defined in Example 6. Standard cathepsin D ELISA determines the levels of cathepsin D in the blood samples prior to affinity separation by the affinity chromatography cartridge. Cathepsin D levels of bound and unbound fractions are assessed.

Affinity Chromatography Cartridge Capture of Galectin-3 from Blood.

Galectin-3 is isolated from normal serum and sera collected from ovarian cancer patients using the optimal affinity chromatography cartridge formulation and procedures established in Example 6. Standard galectin-3 D ELISA determines the levels of galectin-3 in the blood samples prior to affinity separation by the affinity chromatography cartridge. Galectin-3 levels of bound and unbound fractions are assessed.

Affinity Chromatography Cartridge Capture of Exosomes from Blood—Removal of Exosomes from Blood.

Exosomes are isolated from normal serum and sera collected from ovarian cancer patients using centrifugation techniques and compared to the optimal affinity chromatography cartridge formulation and procedures established in Example 6. A total of five aliquots are taken at timeframes determined above that showed 0% (control), 20%, 40%, 60% and >80% tumor exosome binding. Aliquots are tested for exosome protein content as described above. Exosome content of the samples is tested independently using the two-step chromatography/centrifugation described above.

Alternative Affinity Reagents.

The lectins selected for initial studies were chosen because they are known to bind characterized ovarian cancer biomarkers and have been successfully used in the construction of affinity chromatography HEMOPURIFIER® affinity capture cartridges used for the biocapture of virus proteins and viral particles. If these lectins fail to isolate the ovarian cancer markers and exosomes at acceptable levels, other lectins (WGA, MAA, PHA-L)) can be surveyed for biomarker capture. Similarly, if the initial antibodies selected fail to isolate the ovarian cancer markers and exosomes at acceptable levels, other galectin-3, cathepsin D, or tumor-exosome (e.g. MICA, HLA-G) antibodies will be surveyed for biomarker capture.

Alternative Ovarian Cancer Biomarkers.

The affinity chromatography cartridges are validated using three putative ovarian cancer markers (cathepsin D, galectin-3, and exosomes) because these markers are detectable in the blood and have clinical significance. If any of these markers fail to meet specific criteria, alternative ovarian cancer biomarkers (e.g. CA-125, prostasin, osteopontin) will be selected.

### Example 8

### In Vitro Characterization of Immunosuppressive Activities Contained within the Unfractionated, Exosome Depleted, and Affinity Bound Fractions

The relative immunosuppressive activity contained within the unfractionated and affinity-purified samples is determined using NK and T cell cytotoxicity assays. T-cell activities are measured using Jurkat E-61 (human T-cell lymphoma) cells or activated T-cells isolated from huPMBC NOD-SCID mice. NK cell assays are conducted using NK cells isolated from huPMBC NOD-SCID mice.

Growth of Jurkat Cells.

Jurkat cells are maintained in RPMI supplemented with 0.1 mM nonessential amino acids, 1 mM sodium pyruvate, 200 mM L-glutamate, 100 μg·ml⁻¹ streptomycin and 100 IU·ml⁻¹ penicillin in a humidified 5% CO₂ chamber at 37° C.

Isolation of Immune Cells from Mice Spleen.

Spleens are harvested from huPMBC NOD-SCID mice, digested into single cell suspensions and NK cells are isolated from the splenocyte mixture using DX5-conjugated microbeads (Miltenyl Biotec) and cultured in IMDM supplemented with 50 μM BME, 10% FCS and 100 U/ml rIL-2. Purity of samples is confirmed by FACS analysis using a CD49b antibody. T-cells are isolated using a T-cell column loaded with Scrubbed Nylon fiber (Cellular Products) to remove macrophages. Single-cell suspensions are then stimulated with anti-CD3 (1100 ng/ml; clone 145-2c11) plus IL2 (5 ng/ml; Biosource International) and cultured in RPMI supplemented with 10% FCS.

T-Cell Apoptosis Assay.

T-cell apoptosis is assessed using a standard Annexin-V apoptosis assay (Molecular Probes) with either Jurkat or activated T-cell isolated from huPMBC NOD-SCID mice. Cells are co-cultured for 24 hours with escalating concentrations of tumor exosomes derived from OVAR-3 tissue culture supernatants or patient sera. To demonstrate the effect of affinity cartridge depletion of exosomes, Jurkat or activated T-cells are pretreated with various fractions (unfractionated, affinity-bound, affinity unbound) from OVAR-3 tissue culture supernatants, normal sera, ovarian cancer patient sera for 24 hours prior to Annexin-V apoptosis assay.

NK Cell Cytotoxicity Assay.

NK cytotoxicity is determined using a standard chromium release assay (Liu, C., et al., Murine mammary carcinoma exosomes promote tumor growth by suppression of NK cell function. J Immunol, 2006. 176(3):1375-85; Zhang, H. G., et al., Curcumin reverses breast tumor exosomes mediated immune suppression of NK cell tumor cytotoxicity. Biochim Biophys Acta, 2007. 1773(7):1116-23, incorporated by reference in their entireties).

Briefly, spleen NK cells are co-cultured with sodium chromate-labeled YAC-1 lymphoma cells or OVAR-3 cells for 4 hours. Cell lysis is determined by measurement of chromium release into culture supernatants. To examine the effects of exosomes on NK cytotoxic activities ex vivo, NK cells are pretreated with various fractions (unprocessed, affinity-bound, affinity unbound) from OVAR-3 tissue culture supernatants, normal sera, ovarian cancer patient sera for 24 hours prior to incubation with the YAC1 lymphoma or OVAR-3 cells.

NK Cell Proliferation Assay.

The effects of exosomes contained in various fractions on NK cell proliferation are determined using ³H-thymidine incorporation (Loeb, S. and W. J. Catalona, Early versus delayed intervention for prostate cancer: the case for early intervention. Nat Clin Pract Urol, 2007. 4(7):348-9, incorporated by reference in its entirety). NK cells are stimulated with rIL2 (100 U/ml) for various times with or without escalating concentrations of tumor exosomes derived from OVAR-3 tissue culture supernatants or patient sera. Plates are pulsed with 1 uCi of ³H-thymidine and harvested after 14 h. ³H-thymidine incorporation is determined using a scintillation counter.

Modulation of CD3-Zeta Expression on Cultured T-Lymphocytes.

The effects of exosomes contained in various fractions on T-cell function are assayed by analyzing CD3-zeta expression on T-cells from Jurkat E-61 cells or activated T-cell isolated from huPMBC NOD-SCID mice. Cells are co-cultured with various amounts of unprocessed, affinity-bound, or exosome-depleted fractions from OVAR-3 tissue culture supernatants, normal sera, ovarian cancer patient sera for 4 days. Cells are then lyzed. CD3-zeta expression is determined by densitometric analysis of western blots using a monoclonal anti-CD3-zeta antibody (Calbiochem).

### Example 9

### In Vivo Characterization of Tumor Growth and Immunosuppressive Activities Contained within the Unfractionated, Exosome Depleted, and Affinity Bound Fractions

The effects of exosome removal on tumor growth and associated immunosuppressive activity in vivo are tested using human lymphocyte-engrafted, severe combined immunodeficient (hu-PBMC-SCID) mice. Animals are treated with unprocessed, exosome depleted, and affinity bound fractions and the effects on the NK cell and T-cell proliferation and activation are determined. The effects of exosome removal on tumor growth is determined by pretreatment of human ovarian cancer cells with unprocessed and affinity-purified samples prior to injection of cells into hu-PBMC NOD-SCID mice.

The demonstration that the selective removal of tumor-derived exosomes using affinity cartridges is capable of boosting the anti-tumor immune response in vivo is an important step prior to use in humans. The potential benefits of exosome removal on immune function is demonstrated by a study showing that the ex vivo removal of exosomes can reduce the immunosuppressive elements found in cancer-patient blood. Immunocompromised mice engrafted with human peripheral blood mononuclear cells are used in this study (Berney, T., et al., Patterns of engraftment in different strains of immunodeficient mice reconstituted with human peripheral blood lymphocytes. Transplantation, 2001. 72(1):133-40; Sabel, M. S., et al., CTLA-4 blockade augments human T lymphocyte-mediated suppression of lung tumor xenografts in SCID mice. Cancer Immunol Immunother, 2005. 54(10):944-52; Turgeon, N. A., et al., Alloimmune injury and rejection of human skin grafts on human peripheral blood lymphocyte-reconstituted non-obese diabetic severe combined immunodeficient beta2-microglobulin-null mice. Exp Biol Med (Maywood), 2003. 228(9):1096-104, incorporated by reference in their entireties).

Isolation of Human Peripheral Blood Monocytes (huPBMC).

Peripheral blood is drawn from healthy human donors after informed consent and Institutional Review Board Approval. HuPBMC is isolated by density-gradient centrifugation on FicollPlaque (Pharmacia-Biotech) for 30 minutes at 900×g and washed with PBS, and resuspended in PBS at a final concentration of 5×10⁷ cells/ml.

Characterization of Human Peripheral Blood Monocytes (huPBMC).

The phenotype of the huPBMCs is analyzed by flow cytometry using directly conjugated antibodies (FITC-labeled mouse-anti-human CD3, CD8, CD19 and PE-conjugated CD4, CD45RO, and HLA-DR; BD Biosciences) and analyzed using a FACStar Plus flow cytometer.

Transplantation of huPBMCs into NOD-Scid-Mice.

NOD-scid-mice are an immunodeficient mouse strain that lacks T-cell, B-cell, complement, and NK cell activities. Successful engraftment of a functional human immune system using huPBMCs have been established for this mouse strain, and is therefore an ideal choice to monitor the immune response of exosome-containing and exosome-depleted samples in an in vivo setting. For engraftment, 2×10⁶ or 1×10⁷ huPBMCs are injected into 7-week old female NOD-scid mice. Spleens are removed from animals 4-weeks following injections and the levels and activity of T-cells and NK cells are determined as described above.

Effects of In Vivo Administration of Exosomes and Exosomes-Depleted Fractions on Immune Cell Function.

The immunosuppressive effects of exosomes on T-cell and NK cell activities in vivo is demonstrated by treatment of huPMBC NOD-SCID mice with exosomes derived from OVARC-3 cultures or ovarian cancer patient sera. Exosomes (10 μg) are injected i.p. into animals 1 week following huPBMC injections. Exosomes are administered twice weekly for 2 weeks. After 1 additional week, animals are sacrificed and T-cells and NK cells isolated from animal spleens, amounts and percentages determined by FACS analysis, and immune cell activities determined by T-cell apoptosis and NK cytotoxic assays. The ability of the affinity cartridges to deplete the immunosuppressive elements from these fluids is demonstrated by administration of the affinity cartridges unbound and bound fractions using the same protocol described above.

Effects of Exosome Depletion on Tumor Growth.

The immunosuppressive effects of exosomes on tumor growth is demonstrated by treatment of huPMBC NOD-scid mice with exosomes derived from OVARC-3 cultures or ovarian cancer patient sera followed by the implantation of OVARC-3 tumor cells into these animals. Exosomes (10 μg) are injected i.p. into animals 1 week following huPBMC injections. Exosomes are administered twice weekly for 2 weeks. After 1 additional week, animals are injected i.p. with 1×10⁶ OVARC-3 cells. Tumor growth is monitored until tumor sizes reach 200 mm³. Animals are then sacrificed and T-cells and NK cells isolated from animal spleens, amounts and percentages determined by FACS analysis, and immune cell activities determined by T-cell apoptosis and NK cytotoxic assays. Tumors are also removed and analyzed for lymphocyte filtration using immunohistochemistry. The ability of the affinity cartridges to deplete the immunosuppressive elements from these fluids is demonstrated by administration of the unbound and bound fractions using the same protocol described above.

### Example 9B

### Selective Capture of Material from Plasma

Fifty milliliters of fresh frozen human plasma was recirculated over a small GNA affinity matrix column. After washing with normal saline, the cartridge was extracted with 10 ml SDS running buffer. A sample was then run on a 4-20% Tris Glycine gel equilibrated with SDS running buffer.

The data shown in FIG. 12 show a substantial reduction in the plasma proteins relative to the initial plasma sample diluted 70-fold from the stock plasma solution. The primary proteins appear to be human serum albumin and some immunoglobulins in addition to some high MW components that did not enter the gel.

To estimate the approximate extent of protein capture from plasma, the protein captured on the matrix from the initial protein present was estimated by A280 nm standardized against bovine serum albumin and corrected for contributions from SDS and buffers. As shown in table 2, only 0.09% of the input protein was retained.

TABLE 2

| | | Total Protein |
|---|---|---|
| Sample 1 | Protein (mg/ml) | (mg) |
| Zero time | 77.58 | 3879 |
| Extraction | 0.35 | 3.5 |
| | % bound to GNA matrix | 0.09% |

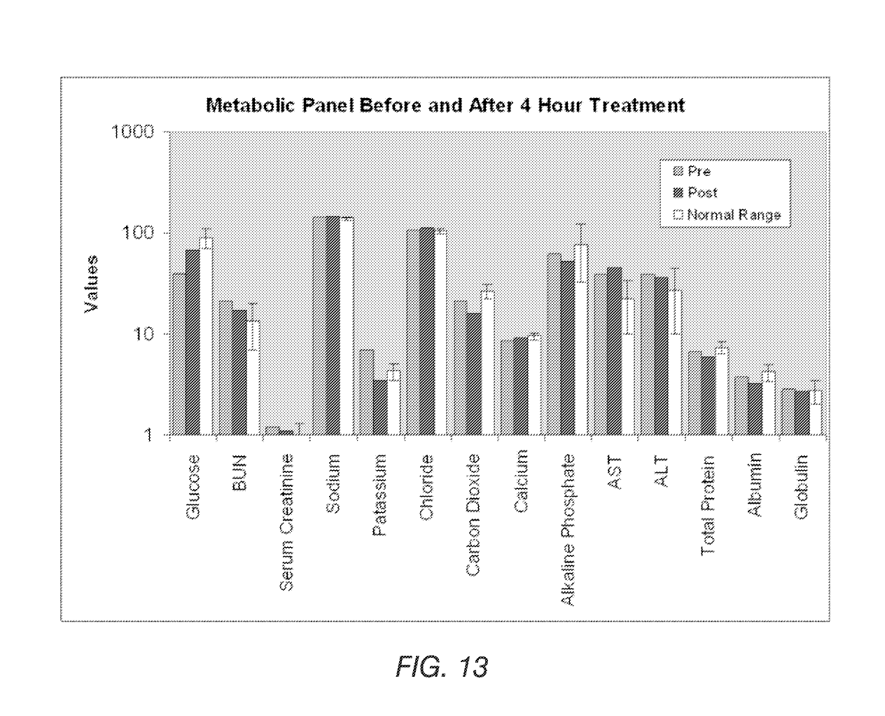

Similar results were found with compounds measured in a standard blood panel. In this experiment, 1 unit of fresh human blood was recirculated over the GNA Hemopurifier at 120 ml per min for 4 hours. Samples were taken prior to the initiation of recirculation (pre) and after completion of the test (post). The samples were then sent to a clinical reference lab for testing. As shown in FIG. 13, the HEMOPURIFIER® cartridge treatment did not seem to significantly change blood chemistry relative to the normal range and the zero time sample.

### Example 10

### Concentration and Total Elution In Vivo

Apollo HCV Safety Trial.

An ERB approved safety study on the HEMOPURIFIER® GNA affinity capture cartridge was carried out in four informed volunteers, each of whom had kidney failure, were on chronic hemodialysis and were infected with Hepatitis C virus (HCV). The study consisted of 3 four hour treatments administered on an every other day schedule. Five cartridges from three patients were tested for HCV virus capture vs. initial viral load (Tullis, R H, R P Duffin, H H Handley, P Sodhi, J Menon, J A Joyce and V Kher (2009). “Reduction of hepatitis c virus using lectin affinity plasmapheresis in dialysis patients.” Blood Purif 27(1): 64-9, incorporated by reference in its entirety).

Briefly, virus extraction was done by first rinsing the cartridges with 1 to 2 liters of sterile saline followed by recirculating 200 ml TriLS to extract and isolate the bound HCV RNA. The isolate was then concentrated by alcohol precipitation and dissolved with 0.5 ml sterile RNase free water. HCV RNA was then quantitated by real-time qRT-PCR using duplicate or triplicate samples. The data are presented in the Table 3.

TABLE 3

| Parameter | Patient Average | TRILS Extract* | Ratio |
|---|---|---|---|
| Total HCV copies | 2,630,824,583 | 889,088,783 | 34% |
| Initial Volume (ml) | 2,746 | 200 | |
| HCV cpm | 957,890 | 13,154,123 | 13.7 |
| Final Volume | 2,746 | 0.5 | |
| HCV cpm in concentrate | 957,890 | 1,778,177,566 | 1856 |

The data show that an average of 34% of the initial HCV virus present in plasma was captured and eluted from the cartridge. The efficiency of extraction was not determined in these experiments. The resultant solution was approximately 14 times more concentrated than the viral RNA in plasma. When this solution was concentrated in alcohol, the final purified solution had an HCV concentration 1856 times higher than the plasma. Given the current PCR detection level for HCV of ˜100 cpm, this result suggests that using this method or purification and concentration of the entire body burden of HCV in plasma would allow detection of 0.054 HCV particles/ml.

Based on the data for non-selective protein binding, we can estimate the extent of purification of the virus relative to protein. Since 34% of the virus was recovered on average vs 0.09% of the protein, the relative purity of the virus has increased 377 fold relative to plasma proteins. Thus the affinity capture has substantially increased both the concentration and relative purity of HCV.

### Example 11

### Concentration and Selective Elution In Vitro GNA Lectin Based Capture and Elution with Mannose

A model virus particle was used. The particle consisted of a 100 nm diameter spherical fluorescent latex bead (Duke Scientific) coated with yeast mannan, a natural mannose polymer. Three ml of 9.5×10⁹ beads/ml (fluorescent mannan coated 100 nm latex beads in PBS) were passed three times over a 0.5 g GNA Chromosorb affinity matrix column in a 3 cm³ column with a glass wool plug. Four different preparations of GNA Chromosorb were used. For these preparations, the average capture of the mannan coated beads was 92%.

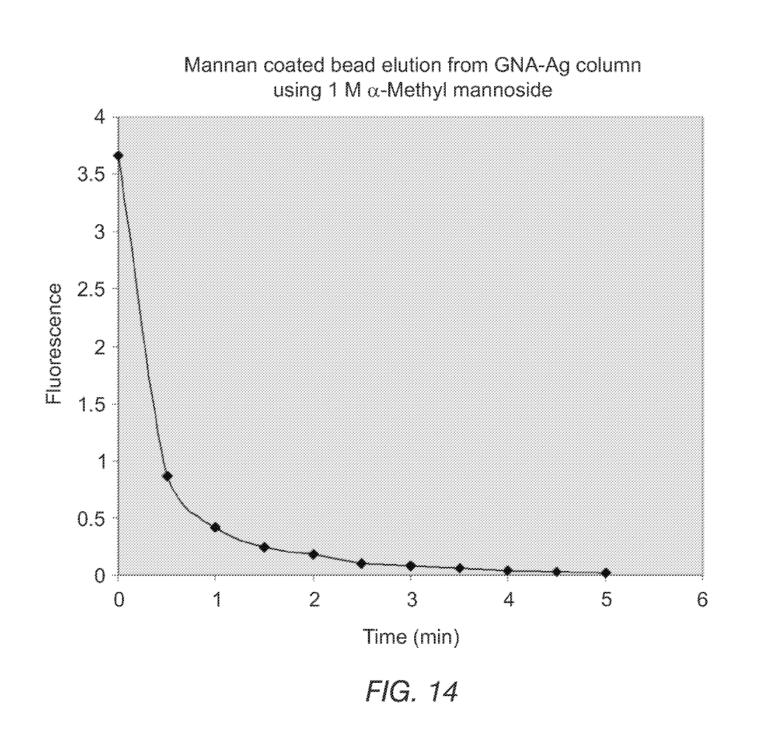

Subsequently, the GNA affinity columns were rinsed with several column volumes of PBS (Phosphate buffered saline) until no further apparent elution was observed. The column was then put on an Aminco Spectrofluorometer equipped with a flow cell and equilibrated with PBS. At zero time, the solvent was switched to 1M α-methylmannoside (AMM) in PBS and the rate of fluorescent bead release monitored at 490 nm (λ_{excitation}=460 nm). In this experiment, the half time for elution was ˜30 seconds. Elution was biphasic when displayed on a log plot. Elution was essentially complete after 5 minutes of washing with AMM. The results are summarized in FIG. 14.

All references cited herein are incorporated herein by reference in their entirety. To the extent publications and patents or patent applications incorporated by reference contradict the disclosure contained in the specification, the specification is intended to supersede and/or take precedence over any such contradictory material.

To the extent publications and patents or patent applications incorporated by reference herein contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein.

Terms and phrases used in this application, and variations thereof, unless otherwise expressly stated, should be construed as open ended as opposed to limiting. As examples of the foregoing, the term ‘including’ should be read to mean ‘including, without limitation’ or the like; the term ‘comprising’ as used herein is synonymous with ‘including,’ ‘containing,’ or ‘characterized by,’ and is inclusive or open-ended and does not exclude additional, unrecited elements or method steps; the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular embodiment of the invention. Likewise, a group of items linked with the conjunction ‘and’ should not be read as requiring that each and every one of those items be present in the grouping, but rather should be read as ‘and/of’ unless expressly stated otherwise. Similarly, a group of items linked with the conjunction ‘or’ should not be read as requiring mutual exclusivity among that group, but rather should be read as ‘and/or’ unless expressly stated otherwise. In addition, as used in this application, the articles ‘a’ and ‘an’ should be construed as referring to one or more than one (for example, to at least one) of the grammatical objects of the article. By way of example, ‘an element’ means one element or more than one element.

The presence in some instances of broadening words and phrases such as ‘one or more’, ‘at least’, ‘but not limited to’, or other like phrases shall not be read to mean that the narrower case is intended or required in instances where such broadening phrases may be absent.

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific embodiments and examples described herein, but rather to also cover all modification and alternatives coming with the true scope and spirit of the invention.

1. (canceled)

2. A method of selectively capturing exosomes that are associated with Chronic Traumatic Encephalopathy (CTE), comprising:
contacting a biological medium obtained from a patient that has CTE with an affinity capture device that comprises a processing chamber configured to receive a biological medium and an affinity capture agent disposed within the processing chamber, wherein said affinity capture agent is a lectin;
capturing exosomes present in the biological medium with the affinity capture agent; and
identifying a biomarker associated with CTE on the captured exosomes.

3. The method of claim 2, wherein the biological medium is selected from the group consisting of blood, serum, plasma, urine, sputum, semen, tissue fluid, and saliva.

4. The method of claim 2, wherein said biomarker is β-amyloid protein.

5. The method of claim 2, wherein said biomarker is tau.

6. The method of claim 2, wherein said lectin is selected from the group consisting of GNA, NPA, and cyanovirin.

7. The method of claim 2, further comprising:
removing the captured exosomes from the affinity capture agent.

8. The method of claim 7, further comprising:
analyzing the removed exosomes by MALDI-TOFF or SELDI-TOFF mass spectrometry or PCR.

9. The method of claim 2, wherein said affinity capture agent is immobilized on a substrate.

10. The method of claim 9, wherein said substrate is a membrane.

11. The method of claim 10, wherein said membrane is a polysulfone, polyethersulfone, polyamide, polyimide, or a cellulose acetate membrane.

12. A method of analyzing the total protein content of captured exosomes comprising:
capturing exosomes present in plasma on an affinity matrix that comprises a lectin; and
determining the total protein present.

13. The method of claim 12, wherein said lectin is GNA.

14. The method of claim 12, wherein said total protein is analyzed by SDS-PAGE.

15. The method of claim 12, wherein said total protein is analyzed at A280 nm.
